(19) United States
(12) Patent Application Publication
(10) Pub. No.: US 2022/0170930 A1
(43) Pub. Date: Jun. 2, 2022

(54) METHOD FOR SCREENING COMPOUND FOR TREATING OR PREVENTING POLYQ-RELATED NEURODEGENERATIVE DISEASES

(71) Applicant: FUDAN UNIVERSITY, Shanghai (CN)

(72) Inventors: Boxun Lu, Shanghai (CN); Yiyan Fei, Shanghai (CN); Yu Ding, Shanghai (CN); Yongjun Dang, Shanghai (CN)

(21) Appl. No.: 17/437,968

(22) PCT Filed: Mar. 11, 2020
(86) PCT No.: PCT/CN2020/078775

§ 371 Date: Sep. 10, 2021

(30) Foreign Application Priority Data
Mar. 11, 2019 (CN) 201910180717.1
Oct. 21, 2019 (CN) 201911000415.8

(51) Int. Cl. G01N 33/566; G01N 33/68; A61K 31/404; A61K 31/352; A61K 31/517; A61P 25/28

(52) U.S. Cl. CPC G01N 33/566; G01N 33/6896; A61P 25/28; A61K 31/352; A61K 31/517; A61K 31/404

(57) ABSTRACT

The present invention relates to a method for screening or identifying compound(s) for the treatment or prevention of a polyQ-related neurodegenerative disorder, and the compounds obtained by the method and uses thereof.

### FIELD OF THE INVENTION

The present invention relates to the biopharmaceutical field, and in particular to a method for screening or identifying compounds for the treatment or prevention of polyQ-related neurodegenerative disorder, and the compounds obtained by the method and uses thereof.

### BACKGROUND OF THE INVENTION

Many diseases are caused by the presence of specific proteins or excessive levels of specific proteins in cells or tissues. The activity of some specific proteins that affect diseases remains unknown. Therefore, for such diseases, the common treatment strategy is to control the protein levels. Some current methods for controlling protein levels, such as biological tools like RNAi, CRIPSR, etc., are difficult to deliver in vivo. Therefore, a feasible treatment strategy is to use low molecular weight compounds (compounds for short) to control the level of proteins that affect diseases. An emerging approach is to enhance the ubiquitination of disease-causing proteins and target them to the proteasome degradation pathway using proteolysis-targeting chimera (PROTAC) technology, but this method relies on certain E3 ligases, which may not be present in diseased cells. Moreover, the protein degradation ability of the proteasome is limited, and the degradation efficiency of certain large disease proteins or aggregates is low.

Neurodegenerative disorder refers to diseases caused by nervous system dysfunction resulted from abnormal death of central neurons. So far, there is no ultimate therapy that can slow down its development. Taking Huntington's disease (HD), the most common neurodegenerative disorder, as an example, it is a monogenetic disorder wherein the mutation in the CAG repeat region of the exon1 of the HTT gene located in the patient's chromosome 4 results in the expansion of the polyglutamine (polyQ) of the synthesized mutant protein (mHTT). mHTT is susceptible to shearing and aggregating, and causes toxicity which eventually leads to dysfunction and death of specific neuron. The current methods for controlling mHTT levels through low molecular weight compounds lack specificity, and may cause side effects. Furthermore, these methods are nonallele-selective and unable to distinguish between mHTT and wild-type HTT protein (wtHTT), which will lead to a decrease in the level of wtHTT which has important biological functions.

Therefore, there is an urgent need for effective screening or identification methods in the art to obtain compounds that can be used to treat or prevent polyQ-related neurodegenerative disorder.

### DISCLOSURE OF THE INVENTION

In one aspect, the present invention provides a method for screening or identifying compound(s) for the treatment or prevention of a polyQ-related neurodegenerative disorder, comprising
(I): bringing the candidate compound(s) into contact with a test system, the test system comprising
   (1) a protein with abnormally expanded polyQ or a fragment thereof containing the polyQ part, and
   (2) a LC3B protein or a homologue thereof or a fragment thereof;

and

(II): determining the ability of the candidate compound(s) to modulate the binding between (1) a protein with abnormally expanded polyQ or a fragment thereof containing the polyQ part, and (2) a LC3B protein, a homologue thereof, or a fragment thereof; and selecting the compound(s) that positively modulate the binding.

In another aspect, the present invention provides the use of the compound obtained by the method of the present invention or the pharmaceutical composition thereof in the preparation of a medicament for the treatment or prevention of a polyQ-related neurodegenerative disorder or use in the preparation of a diagnostic reagent or a kit for detecting subjects considered to be suffering from or susceptible to a polyQ-related neurodegenerative disorder.

In another aspect, the present invention provides the use of a compound or a pharmaceutical composition thereof in the preparation of a medicament for the treatment or prevention of a polyQ-related neurodegenerative disorder; wherein the compound positively modulate the binding between (1) a protein with abnormally expanded polyQ or a fragment thereof containing the polyQ part, and (2) a LC3B protein, a homologue thereof, or a fragment thereof.

### BRIEF DESCRIPTION OF THE DRAWINGS

FIG. 1: Detection of the affinity of the compounds in a system with both LC3B and full-length HTT.

FIG. 2: Effects of compounds on HTT levels in cortical neurons of Hdh^{Q140/Q7} mice.

FIG. 3: Effects of compound 1 and compound 2 on HTT levels in cortical neurons of Hdh^{Q7/Q7} mice.

FIG. 4: Detection of HTT levels in cortical neurons of Hdh^{Q140/Q7} mice using a variety of antibodies after treatment with the compounds.

FIG. 5: Detection of the N-terminal fragments of mHTT in cortical neurons of Hdh^{Q140/Q7} mice by antibodies MW1 and 3B5H10 after treatment with the compounds.

FIG. 6: Cell viability test results of Hdh^{Q140/Q7} mouse cortical neurons after treatment with the compounds.

FIG. 7: Effects of compounds on mHTT levels in primary fibroblasts of HD patients at the concentration of 100 nM.

FIG. 8: Effects of compounds on mHTT levels in immortalized fibroblasts of HD patients.

FIG. 9: Compound 4 reduces mHTT levels in immortalized fibroblasts of HD patients.

FIG. 10: Effects of compounds on mHTT levels in HD patient iPSC-derived neurons.

FIG. 11: Effects of compounds on the apoptosis of HD patient iPSC-derived neurons. The scale bar is 50 μm.

FIG. 12: Effects of compounds on the apoptosis of HD patient iPSC-derived neurons. I, II, III, IV represent compound 1, compound 2, compound 3, compound 4, respectively.

FIG. 13: Effects of compounds on mHTT levels in Huntington's disease Drosophilae.

FIG. 14: Effects of compounds on the survival rate of Huntington's disease Drosophilae. I, II, III, IV represent compound 1, compound 2, compound 3, compound 4, respectively.

FIG. 15: Effects of compounds on the climbing performance of Huntington's disease Drosophilae. I, II, III, IV represent compound 1, compound 2, compound 3, compound 4, respectively.

FIG. 16: Effects of intracerebroventricular injection of compounds on the levels of mHTT and wtHTT in the cortices of Huntington's disease mice.

FIG. 17: Effects of intraperitoneal injection of compounds on the levels of mHTT and wtHTT in the cortices and striata of Huntington's disease mice.

FIG. 18: Detection of mHTT aggregates in the cortices of Huntington's disease mice after intraperitoneal injection of the compound.

FIG. 19a and FIG. 19b: Effects of intraperitoneal injection of compounds on behavioral defects in Huntington's disease mice. I, II, III, IV represent compound 1, compound 2, compound 3, compound 4, respectively.

FIG. 20-1, FIG. 20-2, FIG. 20-3 and FIG. 20-4: The compound does not affect the autophagy function and the levels of control proteins of cultured Hdh^{Q140/Q7} mouse cortical neurons.

FIG. 21: Intraperitoneal injection of the compound does not affect the level of SQSTM1/p62 in mouse cortices.

FIG. 22: The compound selectively enhances the binding between mHTT and LC3B.

FIG. 23: Effects of compounds on the co-localization between mHTT and LC3B in Hela cells. White arrows indicate representative co-localization points. Parts of the images are amplified to show the co-localization points. The scale bar is 10 μm.

FIG. 24: Effects of compounds on the co-localization between mHTT and LC3B in STHdh^{Q111/Q111} cells.

FIG. 25: Effects of compounds on the level of proteins containing polyglutamine in HEK293T cells.

FIG. 26: Effects of compounds on the level of ATXN3 protein in fibroblasts of patients with spinocerebellar ataxia type 3.

FIG. 27: Effects of compounds on the level of mutant ATXN1 protein in HEK293T cells.

### DETAILED DESCRIPTION

The specific embodiments are provided below to illustrate technical contents of the present disclosure. Those skilled in the art can easily understand other advantages and effects of the present disclosure through the contents disclosed in the specification. The present disclosure can also be implemented or applied through other different specific embodiments. Various modifications and variations can be made by those skilled in the art without departing from the spirit of the present disclosure.

### General Terminology and Definitions

Unless defined otherwise, all technical and scientific terms used herein have the same meaning as is commonly understood by one of skill in the art. If there is a contradiction, the definition provided in this application shall prevail. When a trade name is present herein, it refers to the corresponding commodity or the active ingredient thereof. All patents, published patent applications and publications cited herein are incorporated herein by reference.

The terms “including”, “comprising”, “having”, “containing”, or “relating to” and other variants thereof, as used herein, are inclusive or open-ended, and not exclusive of other elements or steps of methods that are not enumerated. Those skilled in the art should understand that the above terms such as “include” also cover the meaning of “consist of”.

The term “one or more” or the similar expression “at least one” can mean, for example, 1, 2, 3, 4, 5, 6, 7, 8, 9, 10 or more.

When the lower limit and the upper limit of a numerical range are disclosed, any numerical value and any included range falling within the range are specifically disclosed. In particular, each value range of the values disclosed herein should be understood to mean each value and range covered in a wider range. For example, the expression “ATXN1 with polyQ length≥40” can cover the case where the polyQ length≥41, and can cover, for example, the case where the polyQ length is 92. For example, “ATXN2 with polyQ length≥33” can cover the case where the polyQ length≥34. For example, “ATXN3 with polyQ length≥41” can cover the case where the polyQ length≥62 and can cover the case where the polyQ length is 74 or 68, for example. For example, “ATXN3 with polyQ length<41” can cover the case where the polyQ length is 27 or 28. For example, “ATXN7 with polyQ length≥19” can cover the case where the polyQ length≥38. For example, “TBP with polyQ length≥44” can cover the case where the polyQ length≥45. For example, “ATN1 with polyQ length≥39” can cover the case where the polyQ length≥49. For example, “HTT with a polyQ length≥36” can cover the case where the polyQ length is 47, 49, 55, 68, 72, 73, 111, 128, or 140. For another example, “HTT with polyQ length<36” can cover the case where the polyQ length is 7, 16, 19, 23, or 25. For example, “AR with polyQ length≥37” can cover the case where the polyQ length≥38.

The term “optional” or “optionally” means that the event or situation described later may or may not occur, and the description includes occurrence of said event or situation and non-occurrence of said event or situation.

The term “neurodegenerative disorder” refers to a disease caused by the loss or pathological change of neurons and/or their myelin sheaths. Characteristic pathological structures, such as insoluble aggregates of protein, can be observed in the brain neurons of patients with neurodegenerative disorders. Insoluble aggregates may produce cytotoxicity, further leading to neuron loss and disease.

The term “polyQ” or “polyglutamine” refers to the polyglutamine tract contained by the protein. Glutamine is encoded by cytosine-adenine-guanine (CAG) in the gene. The length of the polyglutamine is related to the number of CAG repeats in gene exons. Therefore, the increase in the number of CAG repeats in gene exons result in polyglutamine expansions in the synthesized protein. Proteins with abnormally expanded polyQ are known to be associated with some neurodegenerative disorder. As used herein, the gene name can use the form of “Q+number” to indicate the number of CAG repeats in exons, such as Q25 or Q72, which respectively indicate 25 repeats or 72 repeats of CAG in exons. In the protein name, the length of the polyglutamine can be expressed in the form of “Q+number” as above, such as Q27 or Q73, which means that the length of the polyglutamine is 27 Q (glutamine) or 73 Q, respectively. The CAG repetitions or glutamine repetitions indicated in the form of “Q+number” herein are all continuous repetitions. Unless otherwise specified, the length of polyQ as used herein refers to the length of the continuous polyglutamine.

The term “polyQ-related neurodegenerative disorder” refers to neurodegenerative disorder associated with abnormal expansion of polyQ, or neurodegenerative disorder that responds to levels of proteins containing expanded polyQ. Neurodegenerative disorders are a group of disorders with clinical and genetic heterogeneity.

“Normal polyQ” refers to the polyQ of a protein in a normal physiological state that has a length less than a specific number. Correspondingly, “abnormally expanded” means that the polyQ of the protein has a length longer than the normal length. For diseases or pathological conditions, the length of polyQ is longer.

As used herein, “normal polyQ protein” includes polyQ (i.e., its polyQ part) of a normal length.

As used herein, a “protein with abnormally expanded polyQ” includes a polyQ (i.e., its polyQ part) of abnormal length, for example, a polyQ part with the length≥40, ≥33, ≥41, ≥19, ≥46, ≥44, ≥39, ≥36 or ≥37.

As an example, the length of a fragment of a normal or abnormal polyQ protein can be 1% or higher, 2% or higher, 3% or higher, 4% or higher, 5% or higher, 10% or higher, 15% or higher, 20% or higher, 25% or higher, 30% or higher, 35% or higher, 40% or higher, 50% or higher, 60% or higher, 70% or higher, 80% or higher, 90% or higher or 95% or higher of the full length of the corresponding protein, but contains the complete polyQ part of a normal or abnormal length.

In a preferred embodiment, when used in the method of the present invention, especially a high-throughput method, a fragment of a protein with abnormally expanded polyQ can be used. It contains the polyQ part of the protein, and the length can be only 1% or higher, 2% or higher, 3% or higher, 4% or higher of the corresponding protein. In an exemplary embodiment, the “protein with abnormally expanded polyQ” or a fragment thereof used may comprise the amino acid sequence of SEQ ID No: 1, 2 or 3. In another exemplary embodiment, the “normal polyQ protein” or a fragment thereof used may comprise the amino acid sequence of SEQ ID No: 4 or 5. In another exemplary embodiment, the “normal polyQ protein” or a fragment thereof used may comprise the amino acid sequence of SEQ ID No: 7. In another exemplary embodiment, the “normal polyQ protein” or a fragment thereof used may comprise the amino acid sequence of SEQ ID No: 6.

“Normal polyQ protein” or “protein with abnormally expanded polyQ” can be a naturally occurring protein that contains a polyQ part of a normal or abnormal length. For example, it can be from humans (for example, ATXN1, ATXN2, ATXN3, ATXN7, ATXN12, TBP, ATN1, HTT or AR) or from other animals, for example, from mice (for example, mouse HTT protein, whose polyQ can have a length such as 140 or 111), or from insects, fish, rodents, artiodactyls, primates.

“Normal polyQ protein” or “protein with abnormally expanded polyQ” can also be artificially engineered or modified protein, but still has its corresponding polyQ part. Such modification can be carried out for the object of processing, purification, characterization, tracing, etc., for example, adding MBP tag or His tag or fusing with green fluorescent protein, or introducing various amino acid substitutions, additions, or deletions. Such technology is well-known to those skilled in the art. Preferably, such a protein contains the corresponding normal or abnormal polyQ part, and has 15% or higher, 20% or higher, 25% or higher, 30% or higher, 35% or higher, 40% or higher, 50% or higher, 60% or higher, 70% or higher, 80% or higher, 90% or higher, or 95% or higher sequence identity with the corresponding protein with normal or abnormal polyQ. Such a protein can be cut or spliced from the full length or fragment(s) of the corresponding protein with normal or abnormal polyQ, and optionally include modifications, or it can be designed de novo.

As used herein, “a protein containing an expanded polyglutamine” refers to a specific protein wherein the polyglutamine tract is expanded. In some cases, for certain diseases, “expanded polyglutamine” can refer to “abnormal polyQ expansion”. In this case, “protein containing expanded polyglutamine” can have the same meaning as “protein with abnormally expanded polyQ” or “abnormally expanded polyQ protein” described herein. However, in some other cases, the polyQ length of the “expanded polyglutamine” can satisfy both: (1) for some specific diseases, such polyQ is longer than the normal length, which is abnormal expansion; (2) for other specific diseases, the length of such polyQ is normal length and does not involve abnormal expansion. Such “protein containing expanded polyglutamine” can also be understood as “protein with abnormally expanded polyQ” or “abnormally expanded polyQ protein”, which is covered by the scope of the present invention and can be used by the skilled person according to actual needs.

As an example, polyQ-related neurodegenerative disorders include but are not limited to spinocerebellar ataxia (SCA) type 1 (polyQ length≥41), type 2 (polyQ length≥34), and type 3 (polyQ length≥62), type 7 (polyQ length≥38), type 12 (polyQ length≥46), type 17 (polyQ length≥45); and dentatorubral-pallidoluysian atrophy (DRPLA, polyQ length≥49), Huntington's Disease (HD, polyQ length≥36) and spinal-bulbar muscular atrophy (SBMA, polyQ length≥38). These diseases are caused by the expansion of CAG repeat regions on ATXN1, ATXN2, ATXN3, ATXN7, ATXN12, TBP, ATN1, HTT and AR genes, respectively (Lesley Jones et al., DNA repair in the trinucleotide repeat disorders, Lancet Neurol. 2017; 16: 88-96). Among them, spinocerebellar ataxia type 3 (SCA3, also known as Machado-Joseph disease, MJD) is the most common autosomal dominant spinocerebellar ataxia and is second only to HD as a common polyQ-related disease in the world. SCA3 is caused by the abnormally expanded polyQ at the C-terminus of the coded protein ATXN3 resulting from the increase in the number of CAG repeats of the Ataxin-3 gene (ATXN3; also known as the MJD1 gene). Examples of normal polyQ proteins described herein include, but are not limited to, ATXN1 with polyQ length<40, ATXN2 with polyQ length<33, ATXN3 with polyQ length<41, ATXN7 with polyQ length<19, ATXN12 with polyQ length<46, TBP with polyQ length<44, ATN1 with polyQ length<39, HTT with polyQ length<36, and AR with polyQ length<37. Correspondingly, examples of the proteins with abnormally expanded polyQ described herein include, but are not limited to, ATXN1 with polyQ length≥40, ATXN2 with polyQ length≥33, ATXN3 with polyQ length≥41, ATXN7 with polyQ length≥19, and ATXN12 with polyQ length≥46, TBP with polyQ length≥44, ATN1 with polyQ length≥39, HTT with polyQ length≥36, and AR with polyQ length≥37.

Human microtubule-associated protein 1 light chain 3β (MAP1LC3B, LC3B) belongs to the microtubule-associated protein 1 light chain 3 (LC3) family in the Atg8 protein family. LC3B is located on the membranes of pro-autophagosomes and autophagosomes, and can control the formation of autophagosomes. LC3B is a key protein in the process of autophagy.

The form of LC3B protein that can be used includes, for example, LC3B-I and LC3B-II, but it is not limited thereto. For LC3B protein, for example, see Uniprot Accession: Q9GZQ8.

Homologues of a LC3B protein can be used herein, as long as it can be used in the method of the present invention, for example, interacting with an abnormally expanded polyQ protein or a fragment thereof containing the polyQ part.

The homologues of a LC3B protein used herein can be derived from eukaryotes, such as yeast or other non-human animals, such as insects (for example, Drosophila), fish, rodents, artiodactyls, primates, etc. Homologues of a LC3B protein can also be derived from other proteins with similar structure and function, such as LC3A, LC3C, GABARAP and GABARAPL1, but are not limited to thereto. For example, see LC3A (Uniprot Accession: Q9H492-1 and Q9H492-2), LC3C (Uniprot Accession: Q9BXW4), GABARAP (Uniprot Accession: 095166) and GABARAPL1 (Uniprot Accession: Q9HOR8-1 and Q9HOR8-2), but not limited to thereto.

As used herein, a fragment of a LC3B protein or a homologue thereof can also be used, as long as it can be used in the method of the present invention, for example, interacting with an abnormally expanded polyQ protein or the fragment thereof containing the polyQ part. Such a fragment, for example, may have 25% or higher, 30% or higher, 35% or higher, 40% or higher, 50% or higher, 60% or higher, 70% or higher, 80% or higher, 90% or higher, 95% or higher or 100% identity with a LC3B protein or a homologue thereof.

A protein or a fragment thereof can be used to achieve the purpose of the present invention, wherein the protein has identity or homology with a LC3B protein or a homologue thereof, as long as it can be used in the method of the present invention. For example, such protein or fragment interacts with an abnormally expanded polyQ protein or the fragment thereof containing the polyQ part. For example, the said identity may be 25% or higher, 30% or higher, 35% or higher, 40% or higher, 50% or higher, 60% or higher, 70% or higher, 80% or higher, 90% or higher, 95% or higher or 100%.

In an exemplary embodiment, the LC3B protein, the homologue thereof, or the fragment thereof has 25% or higher, 30% or higher, 35% or higher, 40% or higher, 50% or higher, 60% or higher, 70% or higher, 80% or higher, 90% or higher, 95% or higher or 100% sequence identity with the amino acid sequence of SEQ ID NO: 8. Those skilled in the art should understand that this can also mean that the LC3B protein, the homologue thereof, or the fragment thereof may contain such sequences.

Those skilled in the art should also understand that the mature form of the following can be used: a LC3B protein, a homologue thereof, or a fragment thereof, and/or, a protein with abnormally expanded polyQ, or, a fragment thereof containing the polyQ part. For example, the C-terminal amino acids 121-125 in a LC3B protein such as SEQ ID NO: 8 can be clipped to obtain the mature form of the LC3B protein. Such a technical solution is also within the scope of the present invention.

Those skilled in the art should understand that, to be used in the present invention, the LC3B protein, the homologue thereof, or the fragment thereof and/or the protein with abnormally expanded polyQ or the fragment thereof containing the polyQ part can be modified according to actual needs. Such a technical solution is also covered by the scope of the present invention. Such modification may include, but is not limited to, adding tags (such as GST or HIS) or labeling, or substituting, deleting, adding or replacing some of the amino acids. For example, for the convenience of cutting tags, a small amount of modification can be made to the termini of the sequence of SEQ ID NO: 8. For example, the M at the N-terminal of SEQ ID NO: 8 can be replaced with GG, and then the modified or unmodified sequence can be optionally added with a tag such as GST, and used in the present invention. Such a modified sequence, or a technical solution corresponding to a sequence having 25% or higher, 30% or higher, 35% or higher, 40% or higher, 50% or higher, 60% or higher, 70% or higher, 80% or higher, 90% or higher, 95% or higher or 100% sequence identity with the modified sequence, is also within the scope of the present invention.

The term “affinity screening” refers to the process of detecting the binding affinity between a sample and a target. The detection methods used for affinity screening can be, for example, absorbance methods, radiation methods (such as proximity scintillation analysis), fluorescence methods (such as fluorescence resonance energy transfer, fluorescence polarization detection, especially time-dependent fluorescence technology), chemiluminescence methods (such as amplified luminescent proximity homogeneous assay, ALPHAScreen), surface plasmon resonance (SPR, can be implemented, for example, using GE's Biacore series), isothermal titration calorimetry (ITC), micro thermophoresis (MST) or oblique-incidence reflectivity difference.

As used herein, “sequence identity” between two amino acid sequences means the percentage of amino acids that are identical between the sequences. “Sequence homology” means the percentage of amino acids that are identical or represent conservative amino acid substitutions. For sequence comparison, usually a sequence is used as a reference sequence to compare the test sequence with. When using the sequence comparison algorithm, the test and reference sequences are input into the computer. If necessary, the sub-sequence coordinates are specified, and the sequence algorithm program parameters are specified. The percent sequence identity of the test sequence relative to the reference sequence is calculated by the sequence comparison algorithm based on the specified program parameters. Examples of algorithms suitable for determining sequence identity and sequence similarity percentages include but are not limited to BLAST and BLAST 2.0 algorithms. The software to perform BLAST analysis is available from the National Center for Biotechnology Information (NCBI).

The term “target sample” includes various sample types that are obtained from a subject and can be used for diagnostic analysis. The target sample herein can be, for example, any cell sample, biological fluid (including blood, serum, spinal fluid, etc.) or any biopsy sample obtained from a subject's tissue.

Screening Method

The concept of the present invention is based at least in part on the discovery of the following new mechanism: a compound that can bind to both of the key protein LC3B in the autophagy process and the protein with abnormally expanded polyQ can promote the degradation of protein with abnormally expanded polyQ through cell autophagy, so as to achieve the purpose of reducing the level of protein with abnormally expanded polyQ, treating or preventing corresponding diseases.

Therefore, in one aspect, the present invention provides a method for screening or identifying compound(s) for the treatment or prevention of a polyQ-related neurodegenerative disorder, comprising

(I): bringing the candidate compound(s) into contact with a test system, the test system comprising
(1) a protein with abnormally expanded polyQ or a fragment thereof containing the polyQ part, and
(2) a LC3B protein, a homologue thereof, or a fragment thereof;

and

(II): determining the ability of the candidate compound(s) to modulate the binding between (1) a protein with abnormally expanded polyQ or a fragment thereof containing the polyQ part, and (2) a LC3B protein, a homologue thereof, or a fragment thereof; and selecting the compound(s) that positively modulate the binding.

The system includes, but is not limited to, a solution system, a subcellular system, a cell (cell culture) system, a tissue system, an organ system, or an animal system.

In one embodiment, candidate compounds can be provided as a compound library. Such libraries can be commercially available or designed and synthesized. Candidate compounds can include peptides, peptidomimetics and small organic molecules, etc. For example, a candidate compound may be selected from a designed and synthesized compound, a compound with a determined structure in a database (e.g. Pubmed), or a compound synthesized de novo. In a specific embodiment, the candidate compound(s) are selected from small organic molecules.

The term “small organic molecule” or “low molecular weight compound” refers to a molecule that is comparable in size to organic molecules commonly used in drugs. The term excludes biological macromolecules (such as proteins, nucleic acids, etc.), but covers small molecular weight proteins or their derivatives, such as dipeptides, tripeptides, tetrapeptides, pentapeptides, etc.

In a specific embodiment, the size of the small organic molecule is about 100 to about 2000 Da, preferably about 200 to about 1000 Da, for example, about 200 to about 900 Da, about 200 to about 800 Da, about 200 to about 700 Da, about 200 to about 600 Da, about 200 to about 500 Da.

The protein with abnormally expanded polyQ or a fragment thereof containing the polyQ part used in the method of the present invention is determined by the targeted polyQ-related neurodegenerative disorder.

In one embodiment, according to the polyQ-related neurodegenerative disorder, the protein with abnormally expanded polyQ comprises a polyQ part with the polyQ length≥40, ≥33, ≥41, ≥19, ≥46, ≥44, ≥39, ≥36, or >37. In a preferred embodiment, the protein with abnormally expanded polyQ comprises ATXN1 with polyQ length≥40 or a fragment thereof containing the polyQ part. In a preferred embodiment, the protein with abnormally expanded polyQ comprises ATXN2 with a polyQ length≥33 or a fragment thereof containing the polyQ part. In another preferred embodiment, the protein with abnormally expanded polyQ comprises ATXN3 with polyQ length≥41 or a fragment thereof containing the polyQ part. In another preferred embodiment, the protein with abnormally expanded polyQ comprises ATXN7 with a polyQ length≥19 or a fragment thereof containing the polyQ part. In a preferred embodiment, the protein with abnormally expanded polyQ comprises ATXN12 with a polyQ length≥46 or a fragment thereof containing the polyQ part. In another preferred embodiment, the protein with abnormally expanded polyQ comprises TBP with a polyQ length≥44 or a fragment thereof containing the polyQ part. In another preferred embodiment, the protein with abnormally expanded polyQ comprises ATN1 with a polyQ length≥39 or a fragment thereof containing the polyQ part. In a preferred embodiment, the protein with abnormally expanded polyQ comprises HTT with a polyQ length≥36 or a fragment thereof containing the polyQ part. In another preferred embodiment, the protein with abnormally expanded polyQ comprises AR with a polyQ length≥37 or a fragment thereof containing the polyQ part.

The “positive regulation” of binding refers to that, when determined under the same conditions, comparing with the case when the compound is absent, when the compound is present, the binding strength between the proteins or the fragments thereof involved in the binding becomes greater, or the binding amount becomes greater. In one embodiment, the positive modulation of the binding by a compound can be measured at the molecular level, for example, by measuring the increase in binding affinity between proteins or the fragments thereof in the presence of the compound. Positive regulation can also be verified by separately measuring the binding strength (such as affinity) of the compound to the proteins or the fragments thereof involved in the binding. A positive regulation can be determined provided that, the compound has affinity for each of the proteins or the fragments thereof involved in the binding, and at the same time does not have affinity activity or has a lower affinity for the corresponding reference.

The present invention does not specifically limit the implementation method of step (II). In one embodiment, step (II) is implemented in the following manner: respectively in the presence/absence of the candidate compound(s), determining the parameters that characterize the binding strength between (1) a protein with abnormally expanded polyQ or a fragment thereof containing the polyQ part, and (2) a LC3B protein, a homologue thereof, or a fragment thereof; and comparing the parameters obtained in the same test in the presence/absence of the candidate compound(s); and selecting the compound(s) that positively regulates the binding of (1) a protein with abnormally expanded polyQ or a fragment thereof containing the polyQ part, and (2) a LC3B protein, a homologue thereof, or a fragment thereof. Depending on the binding test performed, the above-mentioned “parameters” can vary, but in particular can be, for example, absorbance value, radioactive signal and/or distribution in the sample, fluorescence signal intensity and/or distribution in the sample, heat change, reflected light intensity, reflected light phase change, etc.

In one embodiment, the binding between (1) a protein with abnormally expanded polyQ or a fragment thereof containing the polyQ part, and (2) a LC3B protein, a homologue thereof, or a fragment thereof is affinity binding, and step (II) comprises: respectively in the presence/absence of the candidate compound(s), determining the binding affinity between (1) and (2), and/or, respectively in the presence/absence of the candidate compound(s), determining the binding amount of (1) and (2); and comparing the measured values obtained in the presence/absence of the candidate compound(s); and selecting the candidate compound(s) if the binding of candidate compound(s) to the above-mentioned proteins or fragments thereof changes positively when the candidate compound(s) are present.

In one embodiment, the method for determining the binding between the proteins or fragments thereof in step (II) is selected from in vitro pull-down assay, Split-TEV, co-immunoprecipitation (co-IP), affinity chromatography (AP), complex co-purification, enzyme-linked immunosorbent assay (ELISA), fluorescent molecular sieve (Fluorescence-SEC), yeast two-hybrid (yeast two-hybrid, Y2H), HIP-HOP (haploinsufficiency profiling and homozygous deletion profiling), time-resolved fluorescence resonance energy transfer (TR-FRET), chemiluminescence method, surface plasmon resonance (SPR), isothermal titration calorimetry, micro thermophoresis and any combination thereof. In a preferred embodiment, the method for determining the binding is selected from the group consisting of in vitro pull-down test, co-immunoprecipitation, enzyme-linked immunosorbent assay, fluorescent molecular sieve, time-resolved fluorescence resonance energy transfer, and any combination thereof.

In another embodiment, before step (I), a step of pre-screening the candidate compound(s) for affinity is also included; comprising

(A): detecting the binding affinity of the candidate compound(s) to (1) a protein with abnormally expanded polyQ or a fragment thereof containing the polyQ part, and/or (B): detecting the binding affinity of the candidate compound(s) to (2) a LC3B protein, a homologue thereof, or a fragment thereof;

and (C): selecting the compound(s) that have binding affinity to (1) a protein with abnormally expanded polyQ or a fragment thereof containing the polyQ part and/or (2) a LC3B protein, a homologue thereof, or a fragment thereof.

Wherein step (A) and step (B) can be carried out independently in any order. For example, step (A) and step (B) can be carried out separately in different systems at the same time. Step (A) can be carried out first, then step (B), and vice versa. In a preferred embodiment, the following compound is selected: a compound that have binding affinity to (1) a protein with abnormally expanded polyQ or a fragment thereof containing the polyQ part, and (2) a LC3B protein, a homologue thereof, or a fragment thereof.

As needed, step (A) and step (B) can be independently carried out or not carried out, and can also be carried out at least once in a certain manner each independently (for example, each independently once, twice, 3 times or more times). Such manner can each independently be qualitative or quantitative as needed.

In a preferred embodiment, step (A) may comprise: determining the affinity reaction equilibrium dissociation constant (K_{d}) between the candidate compound(s) and (1) a protein with abnormally expanded polyQ or a fragment thereof containing the polyQ part; and selecting the compound whose affinity reaction equilibrium dissociation constant is 100 μM or less, preferably 10 μM or less, particularly preferably 1 μM or less, such as 600 nM or less, 500 nM or less, 400 nM or less, 300 nM or less, 200 nM or less, 100 nM or less, etc.

In a preferred embodiment, step (B) may comprise: determining the affinity reaction equilibrium dissociation constant between the candidate compound(s) and (2) a LC3B protein, a homologue thereof, or a fragment thereof; and selecting the compound whose affinity reaction equilibrium dissociation constant is 100 μM or less, preferably 10 μM or less, particularly preferably 1 μM or less, such as 600 nM or less, 500 nM or less, 400 nM or less, 300 nM or less, 200 nM or less, 100 nM or less, etc.

In an alternative, step (A) and step (B) are carried out simultaneously in the same system, that is, the candidate compound(s) are brought into contact with (1) a protein with abnormally expanded polyQ or a fragment thereof containing the polyQ part, and (2) a LC3B protein, a homologue thereof, or a fragment thereof at the same time; and detecting the binding affinity of candidate compound(s) to the protein or the fragment thereof, respectively.

In one embodiment, the method for detecting binding affinity in step (A) or step (B) is selected from the group consisting of proximity scintillation analysis, fluorescence resonance energy transfer, fluorescence polarization detection, fluorescent molecular sieves, micro thermophoresis, chemiluminescence, surface plasmon resonance, isothermal titration calorimetry, oblique-incidence reflectivity difference and any combination thereof. In a preferred embodiment, the method for detecting binding affinity in step (A) or step (B) is selected from the group consisting of fluorescence resonance energy transfer, fluorescence polarization detection, oblique-incidence reflectivity difference and any combination thereof.

In a particularly preferred embodiment, the method for detecting the binding affinity in step (A) or step (B) is the oblique-incidence reflectivity difference.

In a preferred embodiment, the step of pre-screening the candidate compound(s) for affinity is performed by high-throughput screening using the oblique-incidence reflectivity difference; comprising:

(a) immobilizing the candidate compound(s) to prepare a compound chip, and scanning the chip to obtain the image before incubation;

(b) incubating the compound chip with the target protein, and scanning the chip to obtain the image after incubation;

wherein the target protein is (1) a protein with abnormally expanded polyQ or a fragment thereof containing the polyQ part or (2) a LC3B protein, a homologue thereof, or a fragment thereof; and

(c) analyzing the difference image (the image after incubation minus the image before incubation); and selecting the compound(s) that can bind to both (1) and (2).

In another embodiment, the method used in step (A) or step (B) to determine the equilibrium dissociation constant is selected from surface plasmon resonance, isothermal titration calorimetry and micro thermophoresis.

In another embodiment, the method of the present invention may further include step (D): detecting the binding of the candidate compound(s) to a normal polyQ protein or a fragment thereof containing the polyQ part. This step may be carried out, for example, after step (II), or after step (C).

In one embodiment, the binding is binding affinity. In one embodiment, a compound that does not bind to normal polyQ protein or a fragment thereof containing the polyQ part is selected. Step (D) can be carried out or not carried out as needed, or carried out at least once (for example, once, twice, three times or more). When carried out more than once, the normal polyQ polypeptide used each time can be independently normal polyQ protein or a fragment thereof containing the polyQ part.

The normal polyQ protein used in step (D) is the normal polyQ protein corresponding to the protein with abnormally expanded polyQ used in step (I). In one embodiment, the protein with abnormally expanded polyQ used in step (I) comprises HTT with abnormally expanded polyQ or a fragment thereof containing the polyQ part. Correspondingly, if step (D) is present, the normal polyQ protein used therein includes normal HTT or a part thereof containing polyQ. In an exemplary embodiment, the protein with abnormally expanded polyQ used in step (I) is HTT with abnormally expanded polyQ or a fragment thereof containing the polyQ part, for example, HTT with polyQ length≥36 or a fragment thereof containing the polyQ part, such as the amino acid sequence of SEQ ID NO: 1 (HTTexon1-Q72). Correspondingly, if step (D) is present, the normal polyQ protein used is HTT with normal polyQ length or a fragment thereof containing the polyQ part, for example, HTT with polyQ length<36 or a fragment thereof containing the polyQ part, such as the amino acid sequence of SEQ ID NO: 4 (HTT-Q23) or the amino acid sequence of SEQ ID NO: 5 (HTTexon1-Q25).

In another embodiment, the protein with abnormally expanded polyQ used in step (I) comprises ATXN3 with abnormally expanded polyQ or a fragment thereof containing the polyQ part. Correspondingly, if step (D) is present, the normal polyQ protein used therein contains normal ATXN3 or a part thereof containing polyQ. In an exemplary embodiment, the protein with abnormally expanded polyQ used in step (I) is ATXN3 with abnormally expanded polyQ or a fragment thereof containing the polyQ part, for example, ATXN3 with a polyQ length≥41 or a fragment thereof containing the polyQ part, such as the amino acid sequence of SEQ ID NO: 2 (ATXN3-Q74). Correspondingly, if step (D) is present, the normal polyQ protein used is ATXN3 with normal polyQ length or a fragment thereof containing the polyQ part, for example, ATXN3 with polyQ length<41 or a fragment thereof containing the polyQ part, such as amino acid sequence of SEQ ID NO: 7 (ATXN3-Q27).

In another embodiment, the protein with abnormally expanded polyQ used in step (I) comprises ATXN1 with abnormally expanded polyQ or a fragment thereof containing the polyQ part. Correspondingly, if step (D) is present, the normal polyQ protein used therein comprises normal ATXN1 or a part thereof containing polyQ. In an exemplary embodiment, the protein with abnormally expanded polyQ used in step (I) is ATXN1 with abnormally expanded polyQ or a fragment thereof containing the polyQ part, for example, ATXN1 with polyQ length≥40 or a fragment thereof containing the polyQ part, such as the amino acid sequence of SEQ ID NO: 3 (ATXN1-Q92). Correspondingly, if step (D) is present, the normal polyQ protein used is ATXN1 with normal polyQ length or a fragment thereof containing the polyQ part, for example, ATXN1 with polyQ length<40 or a fragment thereof containing the polyQ part.

In another embodiment, the protein with abnormally expanded polyQ used in step (I) comprises polyQ of abnormal length, which is labeled with a detectable molecule. In one embodiment, the protein with abnormally expanded polyQ used in step (I) is a fusion protein of fluorescent protein (such as GFP or YFP) and polyQ with abnormal length. In a preferred embodiment, the protein with abnormally expanded polyQ used in step (I) is a polyQ-GFP protein containing polyQ with abnormal length, preferably selected from Q72-GFP, Q53-GFP, Q46-GFP. According to the specific disease targeted by the screening, in an alternative embodiment, the protein with abnormally expanded polyQ used in step (I) is Q38-GFP.

In another embodiment, the normal polyQ protein used in step (D) contains polyQ of normal length, which is labeled with a detectable molecule. In one embodiment, the normal polyQ protein used in step (D) is a fusion protein of fluorescent protein (such as GFP or YFP) and polyQ with normal length. In a preferred embodiment, the normal polyQ protein used in step (D) is a polyQ-GFP protein containing polyQ of normal length, preferably Q25-GFP. According to the specific disease targeted by the screening, in an alternative embodiment, the normal polyQ protein used in step (D) is Q38-GFP.

In one embodiment, the LC3B protein, the homologue thereof, or the fragment thereof have 25% or higher, 30% or higher, 35% or higher, 40% or higher, 50% or higher, 60% or higher, 70% or higher, 80% or higher, 90% or higher, 95% or higher or 100% sequence identity with the amino acid sequence of SEQ ID NO: 8. In a preferred embodiment, the homologue of the LC3B protein comes from Drosophila or mouse.

In a particular embodiment, the LC3B protein homologue is the Atg8 protein in Drosophila (31% sequence identity with human LC3B).

In yet another aspect, the present invention also provides a method for screening or identifying compound(s) for the treatment or prevention of a polyQ-related neurodegenerative disorder, comprising

(1): determining the effect of the candidate compound(s) on the level of protein with abnormally expanded polyQ in the cell; and selecting the compound(s) that can reduce the level of the protein; or

(2): determining the effect of candidate compound(s) on the subcellular localization of the protein with abnormally expanded polyQ or a fragment thereof containing the polyQ part in the cell; and selecting the compound(s) that can promote the protein with abnormally expanded polyQ or the fragment thereof containing the polyQ part to localize to cellular substructures with autophagy function such as autophagosomes; preferably, a compound that can promote the co-localization between polyQ abnormal expansion protein or a fragment thereof containing the polyQ part and LC3B protein is selected,

wherein the terms such as protein with abnormally expanded polyQ, fragment thereof containing the polyQ part, and LC3B protein are as defined above.

In one embodiment, in step (1) the following compound is selected: a compound that can reduce the level of the protein with abnormally expanded polyQ by more than 10% in the cell, preferably 20% or more, such as about 20%, about 30%, about 40%, about 50%, etc.

The term “polypeptide” refers to a polymer of amino acids of a certain length. Therefore, peptides, oligopeptides and proteins are included in the definition of “polypeptide”, and these terms are used interchangeably herein. The term “polypeptide” or “protein” does not exclude post-translational modifications, including but not limited to phosphorylation, acetylation, glycosylation, etc. The protein or protein fragment of the present invention can be produced by any technique known in the art per se, such as but not limited to any chemical, biological, genetic or enzymatic technique used alone or in combination.

Knowing the amino acid sequence of the desired sequence, those skilled in the art can easily prepare the protein or protein fragment by standard techniques for producing the protein or protein fragment. For example, they can be synthesized using the well-known solid-phase method, preferably using a commercially available peptide synthesis equipment (such as those prepared by Applied Biosystems, Foster City, Calif.) and following the manufacturer's instructions.

Alternatively, the protein or protein fragment of the present invention can be synthesized by the well-known recombinant DNA technology in the art. For example, after the DNA sequence encoding the desired (poly)peptide is incorporated into an expression vector and this vector is introduced into a suitable eukaryotic or prokaryotic host for expressing the desired protein or protein fragment, these fragments can be obtained as DNA expression products, and then they can be isolated from the host using well-known techniques.

A variety of host/expression vector combinations can be used to express nucleic acids encoding proteins or protein fragments of the invention. Useful expression vectors include, for example, segments of chromosomal, non-chromosomal and synthetic DNA sequences.

Suitable vectors include but are not limited to derivatives of SV40 and pcDNA; and known bacterial plasmids, such as col EI, pCR1, pBR322, pMal-C2, pET, pGEX, pMB9 and derivatives thereof; plasmids, such as RP4; phage DNAs, such as the numerous derivatives of phage I, such as NM989, and other phage DNA, such as M13 and filamentous single-stranded phage DNA; yeast plasmids, such as the 2 microns plasmid or derivatives of the 2 microns plasmid, as well as centromeres and integrative yeast shuttle vectors; vectors useful in eukaryotic cells, such as vectors useful in insect or mammalian cells; vectors derived from combinations of plasmids and phage DNAs, such as plasmids that have been modified to employ phage DNA or the expression control sequences; etc.

Therefore, mammalian and typical human cells, as well as bacterial, yeast, fungal, insect, nematode and plant cells can be used in the present invention and can be transfected by the nucleic acid or recombinant vector defined herein. Examples of suitable cells include, but are not limited to, VERO cells; HELA cells, such as ATCC No. CCL2; CHO cell lines, such as ATCC No. CCL61; COS cells, such as COS-7 cells and ATCC No. CRL 1650 cells; W138, BHK, HepG2, 3T3, such as ATCC No. CRL6361; A549, PC12, K562 cells, 293T cells, Sf9 cells, such as ATCC No. CRL1711 and Cv1 cells, such as ATCC No. CCL70. Other suitable cells that can be used in the present invention include but are not limited to prokaryotic host cell strains, for example, Escherichia coli (e.g., strain DH5-[α]), Bacillus subtilis, Salmonella typhimurium or strains of the genera of Pseudomonas, Streptomyces and Staphylococcus. Other suitable cells that can be used in the present invention include yeast cells, such as Saccharomyces cells, such as Saccharomyces cerevisiae.

Those skilled in the art can understand that the protein or protein fragment of the present invention can be modified for purposes such as facilitating experimental operations, increasing solubility, facilitating crystallization and purification, but does not affect the function of the protein or protein fragment. Such proteins or protein fragments and use thereof also belong to the scope of the present invention.

In one embodiment, (1) a protein with abnormally expanded polyQ or a fragment thereof containing the polyQ part or (2) a LC3B protein, a homologue thereof, or a fragment thereof can be labeled with detectable molecules for screening purposes.

The detectable molecule can be composed of any compound or substance that can be detected by spectroscopic, photochemical, biochemical, immunochemical, or chemical means. For example, useful detectable molecules include radioactive substances (including those containing 32P, 25S, 3H, or 125I), fluorescent dyes (including 5-bromodeoxyuridine, fluorescein, acetamidofluorene or digitoxin), fluorescent proteins (such as GFP and YFP, where GFP can be sfGFP, for example).

In one embodiment, the detectable label is located on or bind to the amino acid residues outside the sequence of (1) a protein with abnormally expanded polyQ or a fragment thereof containing the polyQ part or (2) a LC3B protein, a homologue thereof, or a fragment thereof; and thereby minimizing or preventing any artificial influence on the binding between the proteins or protein fragments or between the candidate compound(s) and any of the proteins or protein fragments.

In a specific embodiment, the protein or protein fragment of the present invention is fused to a fluorescent protein such as a GFP tag (green fluorescent protein). In another specific embodiment, the protein or protein fragment of the present invention is labeled respectively with a suitable fluorescent group in a manner suitable for fluorescence energy transfer analysis.

Regardless of the implementation methods of steps (I), (II), (A), (B), (C), (b), step (1) or step (2) of the screening method of the present invention, a complete protein with abnormally expanded polyQ and a complete LC3B protein or a homologue thereof can be used for the assay. Alternatively, a fragment of a protein with abnormally expanded polyQ or a fragment of LC3B protein or a homologue thereof can be used in the assay; wherein the said fragment includes the binding site.

In a further aspect, the present invention provides a screening system comprising more than two parts, wherein part (A) contains an unlabeled or suitably labeled protein with abnormally expanded polyQ or a fragment thereof containing the polyQ part, and part (B) contains an unlabeled or suitably labeled LC3B protein, a homologue thereof, or a fragment thereof. Each part is independently isolated or not isolated from the other. Each part independently optionally contains suitable buffer(s) and/or detection reagent(s). In a specific embodiment, the screening system of the present invention is in the form of a kit. In another specific embodiment, the prepared compound chip is placed in the kit of the present invention, after incubating with (1) a protein with abnormally expanded polyQ or a fragment thereof containing the polyQ part, and (2) a LC3B protein, a homologue thereof, or a fragment thereof, the signal is analyzed with a suitable plate reader, and compounds that can bind to both (1) and (2) are obtained from screening.

Compounds of the Invention

The present invention also relates to compounds obtained by the method of the present invention. The compounds of the present invention encompass the pharmaceutically acceptable salts, stereoisomers, solvates, polymorphs, tautomers, isotopic compounds, metabolites or prodrugs thereof.

The term “pharmaceutically acceptable” refers to that when contacted with the patient's tissue within the scope of normal medical judgment, no undue toxicity, irritation, allergic reactions, etc. shall arise.

The pharmaceutically acceptable salts of the compounds of the present invention include acid addition salts and base addition salts thereof. The method for preparing the pharmaceutically acceptable salt of the compound of the present invention is known to those skilled in the art.

The compounds of the present invention may exist in specific geometric or stereoisomeric forms, including cis and trans isomers, (−)- and (+)-enantiomers, (R)- and (S)-enantiomers, diastereomers, (D)-isomers, (L)-isomers, and their racemic mixtures and other mixtures, for example, mixtures enriched in enantiomers or diastereomers, all of which are within the scope of the present invention.

The compounds of the present invention may exist in the form of solvates (preferably hydrates), wherein the compound of the present invention contains a polar solvent as a structural element of the compound crystal lattice, especially for example water, methanol, or ethanol. The amount of polar solvents, especially water, can be present in stoichiometric or non-stoichiometric ratios.

The present invention also covers all possible crystalline forms or polymorphs of the compounds of the present invention, which can be a single polymorph or a mixture of more than one polymorph in any ratio.

The metabolites of the compounds of the present invention are also included within the scope of the present invention, that is, substances formed in the body when the compounds of the present invention are administered. Such products can be produced, for example, by oxidation, reduction, hydrolysis, amidation, deamidation, esterification, enzymatic hydrolysis, etc. of the administered compound.

The present invention further includes within its scope the prodrugs of the compounds of the present invention, which are certain derivatives of the compounds of the present invention that have less or no pharmacological activity themselves but when administered to or on the body, can be converted into the compound of the present invention with the desired activity by, for example, hydrolytic cleavage.

The term “polymorphism” or “polymorph” refers to a single polymorph or a mixture of more than one polymorph in any ratio.

The term “crystal form” or “crystal” refers to any solid substance exhibiting a three-dimensional order, as opposed to an amorphous solid substance, which produces a characteristic X-ray powder diffraction pattern with sharp and defined peaks.

The term “amorphous” refers to any solid substance which lacks order in three dimensions.

The compound of the present invention can be applied in the form of a pharmaceutical composition. The pharmaceutical composition comprises the compound or a pharmaceutically acceptable salt, stereoisomer, solvate, polymorph, tautomer, isotopic compound, metabolite or prodrug thereof, and at least one pharmaceutical acceptable carrier.

The term “pharmaceutically acceptable carrier” refers to those substances that have no obvious stimulating effect on the organism and will not damage the biological activity and performance of the active compound. “Pharmaceutically acceptable carriers” include but are not limited to glidants, sweeteners, diluents, preservatives, dyes/colorants, flavors, surfactants, wetting agents, dispersants, disintegrants, stabilizer, solvent or emulsifier.

In one aspect, the present invention provides the use of the compound of the present invention or the pharmaceutical composition thereof in the preparation of a medicament for the treatment or prevention of a polyQ-related neurodegenerative disorder.

In another aspect, the present invention provides compounds of the present invention for use in the treatment or prevention of a polyQ-related neurodegenerative disorder.

In another aspect, the present invention provides a method of treating or preventing a polyQ-related neurodegenerative disorder, comprising administering a compound of the present invention to an individual in need thereof.

In another aspect, the present invention provides use of a compound or a pharmaceutical composition thereof that positively modulates the binding between (1) a protein with abnormally expanded polyQ or a fragment thereof containing the polyQ part, and (2) a LC3B protein, a homologue thereof, or a fragment thereof in the preparation of a medicament for the treatment or prevention of a polyQ-related neurodegenerative disorder.

In another aspect, the present invention provides a compound or a pharmaceutical composition thereof that positively regulates the binding between (1) a protein with abnormally expanded polyQ or a fragment thereof containing the polyQ part, and (2) a LC3B protein, a homologue thereof, or a fragment thereof; for treating or preventing a polyQ-related neurodegenerative disorder.

In another aspect, the present invention provides a method of treating or preventing a polyQ-related neurodegenerative disorder, including administering a compound or a pharmaceutical composition thereof that positively regulates the binding between (1) a protein with abnormally expanded polyQ or a fragment thereof containing the polyQ part, and (2) a LC3B protein, a homologue thereof, or a fragment thereof, to individuals in need thereof.

The said compound that positively regulates the binding between (1) a protein with abnormally expanded polyQ or a fragment thereof containing the polyQ part, and (2) a LC3B protein, a homologue thereof, or a fragment thereof can be obtained, for example, by the method of the present invention.

In a preferred embodiment, the polyQ-related neurodegenerative disorder is selected from spinocerebellar ataxia type 1 (SCA1), spinocerebellar ataxia type 2 (SCA2), spinocerebellar ataxia type 3 (SCA3), spinocerebellar ataxia type 7 (SCA7), spinocerebellar ataxia type 12 (SCA12), spinocerebellar ataxia type 17 (SCA17), dentatorubral-pallidoluysian atrophy (DRPLA), Huntington's disease (HD) and spinal-bulbar muscular atrophy (SBMA), especially Huntington's disease and spinocerebellar ataxia type 3. In a more preferred embodiment, the polyQ-related neurodegenerative disorder is selected from Huntington's disease and SCA3; especially Huntington's disease.

In a further aspect, the present invention provides an article of manufacture, for example in the form of a kit. The article of manufacture of the present invention contains the compound or the pharmaceutical composition of the present invention, and optionally includes a packaging material and a package insert (instructions).

In yet another aspect, the present invention provides the use of the compounds of the present invention for screening or identifying target proteins that can be degraded through the autophagy pathway. In one embodiment, for example, the compound obtained from screening can be used to find the target protein by the following method. 1) Screening or identifying proteins that can bind to the compound in affinity; and 2) determining the ability of the compound to modulate the binding between the protein obtained in 1) and a LC3B protein, a homologue thereof, or a fragment thereof; wherein, if the compound can positively regulate the binding, the protein obtained in 1) can be degraded through the autophagy pathway.

In another aspect, the present invention provides the use of the compound of the present invention or a pharmaceutical composition thereof in the preparation of a diagnostic reagent or a kit for detecting subjects considered to be suffering from or susceptible to a polyQ-related neurodegenerative disorder. In one embodiment, the aforementioned test includes the step of analyzing a target sample obtained from the subject, comprising:

i) labeling the compound with a detectable label such as a fluorescent group;

ii) treating the target sample with the compound obtained in i);

iii) detecting the position and/or intensity of the detectable signal in the target sample; and obtaining the position and/or content of the protein with abnormally expanded polyQ or the fragment thereof containing the polyQ part in the target sample.

In a preferred embodiment, the target sample is selected from cell samples, blood, serum, spinal fluid; or any biopsy sample obtained from the subject's tissue.

In a further aspect, the present invention provides a detection kit. The kit contains the compound of the present invention or a pharmaceutical composition thereof, and optionally contains reagent(s) required for detection, such as aqueous solutions, solvents, suitable detection reagent(s) such as chemiluminescence reagent(s), etc.

The invention also relates to compounds selected from:

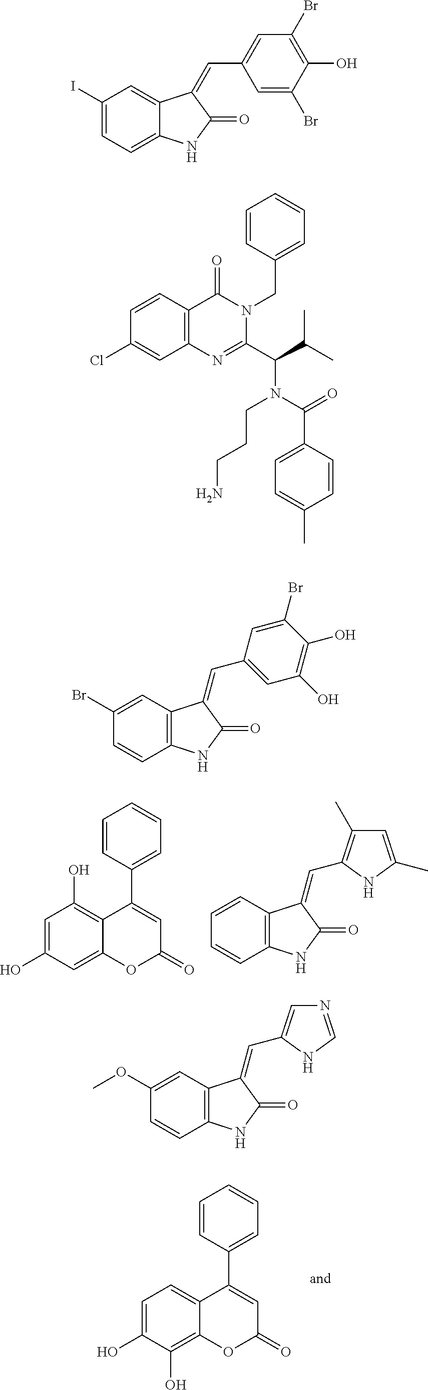

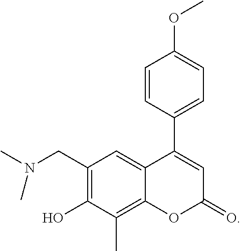 [SMILES: COc1ccc(-c2cc(=O)oc3c(C)c(O)c(CN(C)C)cc23)cc1]

As mentioned above, the embodiments of the present invention can be listed as follows:

[1] A method for screening or identifying compound(s) for the treatment or prevention of a polyQ-related neurodegenerative disorder, comprising

(I): bringing the candidate compound(s) into contact with a test system, the test system comprising
(1) a protein with abnormally expanded polyQ or a fragment thereof containing the polyQ part, and
(2) a LC3B protein, a homologue thereof, or a fragment thereof;

and

(II): determining the ability of the candidate compound(s) to modulate the binding between (1) a protein with abnormally expanded polyQ or a fragment thereof containing the polyQ part, and (2) a LC3B protein, a homologue thereof, or a fragment thereof and selecting the compound(s) that positively modulate the binding.

[2] The method of [1] above, wherein step (II) comprises:

determining the binding affinity between (1) a protein with abnormally expanded polyQ or a fragment thereof containing the polyQ part, and (2) a LC3B protein, a homologue thereof, or a fragment thereof in the presence/absence of the candidate compound(s); and/or

determining the binding amount of (1) a protein with abnormally expanded polyQ or a fragment thereof containing the polyQ part, and (2) a LC3B protein, a homologue thereof, or a fragment thereof in the presence/absence of the candidate compound(s).

[3] The method of [1] or [2] above, wherein the protein with abnormally expanded polyQ comprises a polyQ part of polyQ length≥40, ≥33, ≥41, ≥19, ≥46, ≥44, ≥39, ≥36, or ≥37;

preferably, the protein with abnormally expanded polyQ comprises

ATXN1 with polyQ length≥40 or a fragment thereof containing the polyQ part,

ATXN2 with polyQ length≥33 or a fragment thereof containing the polyQ part,

ATXN3 with polyQ length≥41 or a fragment thereof containing the polyQ part,

ATXN7 with polyQ length≥19 or a fragment thereof containing the polyQ part,

ATXN12 with polyQ length≥46 or a fragment thereof containing the polyQ part,

TBP with polyQ length≥44 or a fragment thereof containing the polyQ part,

ATN1 with polyQ length≥39 or a fragment thereof containing the polyQ part,

HTT with polyQ length≥36 or a fragment thereof containing the polyQ part, or

AR with polyQ length≥37 or a fragment thereof containing the polyQ part.

[4] The method of any one of the above [1] to [3], wherein (2) a LC3B protein, a homologue thereof, or a fragment thereof has 25% or higher, 30% or higher, 35% or higher, 40% or higher, 50% or higher, 60% or higher, 70% or higher, 80% or higher, 90% or higher, 95% or 100% sequence identity with the amino acid sequence of SEQ ID NO: 8.

[5] The method of any one of [1] to [4] above, wherein the method for determining the binding is selected from the group consisting of in vitro pull-down test, Split-TEV, co-immunoprecipitation, affinity chromatography, complex co-purification, enzyme-linked immunosorbent assay, fluorescent molecular sieve, yeast two-hybrid, HIP-HOP, time-resolved fluorescence resonance energy transfer, chemiluminescence method, surface plasmon resonance, isothermal titration calorimetry, micro thermophoresis and any combination thereof; preferably, the method for determining the binding is selected from the group consisting of in vitro pull-down experiments, co-immunoprecipitation, enzyme-linked immunosorbent assay, fluorescent molecular sieves, time-resolved fluorescence resonance energy transfer, and any combination thereof.

[6] The method of any one of [1] to [5] above, wherein before step (I), it further includes a step of pre-screening the candidate compound(s) for affinity, comprising:

(A): detecting the binding affinity of the candidate compound(s) to (1) a protein with abnormally expanded polyQ or a fragment thereof containing the polyQ part, and/or (B): detecting the binding affinity of the candidate compound(s) to (2) a LC3B protein, a homologue thereof, or a fragment thereof; and

(C): selecting the compound(s) that have binding affinity to (1) a protein with abnormally expanded polyQ or a fragment thereof containing the polyQ part and/or (2) a LC3B protein, a homologue thereof, or a fragment thereof;

wherein step (A) and step (B) can be carried out independently in any order.

[7] The method of [6] above, wherein

step (A) comprises: determining the affinity reaction equilibrium dissociation constant between the candidate compound(s) and (1) a protein with abnormally expanded polyQ or a fragment thereof containing the polyQ part; and selecting the compound(s) whose affinity reaction equilibrium dissociation constant is 100 μM or less, preferably 10 μM or less, particularly preferably 1 μM or less; and/or

step (B) comprises: determining the affinity reaction equilibrium dissociation constant between the candidate compound(s) and (2) a LC3B protein, a homologue thereof, or a fragment thereof; and selecting the compound(s) whose affinity reaction equilibrium dissociation constant is 100 μM or less, preferably 10M or less, particularly preferably 1 μM or less.

[8] The method of [6] or [7] above, wherein the method for determining binding affinity is selected from the group consisting of proximity scintillation analysis, fluorescence resonance energy transfer, fluorescence polarization detection, fluorescent molecular sieves, micro thermophoresis, chemiluminescence, surface plasmons resonance, isothermal titration calorimetry, oblique-incidence reflectivity difference and any combination thereof; Preferably selected from the group consisting of fluorescence resonance energy transfer, fluorescence polarization detection, oblique-incidence reflectivity difference and any combination thereof; particularly preferably oblique-incidence reflectivity difference.

[9] The method of any one of [6]-[8] above, wherein

the step of pre-screening the candidate compound(s) for affinity is performed by high-throughput screening using the oblique-incidence reflectivity difference; comprising:

(a) immobilizing the candidate compound(s) candidate compound(s) to prepare a compound chip, and scanning the chip to obtain the image before incubation;

(b) incubating the compound chip with the target protein, and scanning the chip to obtain the image after incubation;

wherein the target protein is (1) a protein with abnormally expanded polyQ or a fragment thereof containing the polyQ part or (2) a LC3B protein, a homologue thereof, or a fragment thereof; and

(c) analyzing the difference image (the image after incubation minus the image before incubation); and selecting the compound(s) that can bind to (1) a protein with abnormally expanded polyQ or a fragment thereof containing the polyQ part, and (2) a LC3B protein, a homologue thereof, or a fragment thereof.

[10] The method of any one of [1]-[9] above further comprises the following steps:

(D): detecting the binding of the candidate compound(s) to a normal polyQ protein or a fragment thereof containing the polyQ part and selecting the compound(s) that do not bind to the normal polyQ protein or the fragment thereof containing the polyQ part.

[11] Use of the compound obtained by the method of any one of the above [1]-[10] or the pharmaceutical composition thereof in the preparation of a medicament for the treatment or prevention of a polyQ-related neurodegenerative disorder, or use in the preparation of diagnostic a reagent or a kit for detecting subjects considered to be suffering from or susceptible to a polyQ-related neurodegenerative disorder;

Preferably, the polyQ-related neurodegenerative disorder is selected from the group consisting of spinocerebellar ataxia type 1, spinocerebellar ataxia type 2, spinocerebellar ataxia type 3, spinocerebellar ataxia type 7, and spinocerebellar ataxia type 12, spinocerebellar ataxia type 17, dentatorubral-pallidoluysian atrophy, Huntington's disease, and spinal-bulbar muscular atrophy, especially Huntington's disease and spinocerebellar ataxia type 3.

[12] The use of [11] above, wherein the detection includes the step of analyzing a target sample obtained from the subject, comprising

i) labeling the compound with a detectable label such as a fluorescent group;

ii) treating the target sample with the compound obtained in i);

iii) detecting the position and/or intensity of the detectable signal in the target sample and obtain the position and/or content of the protein with abnormally expanded polyQ or the fragment thereof containing the polyQ part in the target sample.

[13] Use of the compound or a pharmaceutical composition thereof in the preparation of a medicament for the treatment or prevention of a polyQ-related neurodegenerative disorder, or use in the preparation of a diagnostic reagent or a kit for detecting subjects considered to be suffering from or susceptible to a polyQ-related neurodegenerative disorder, wherein the compound is selected from:

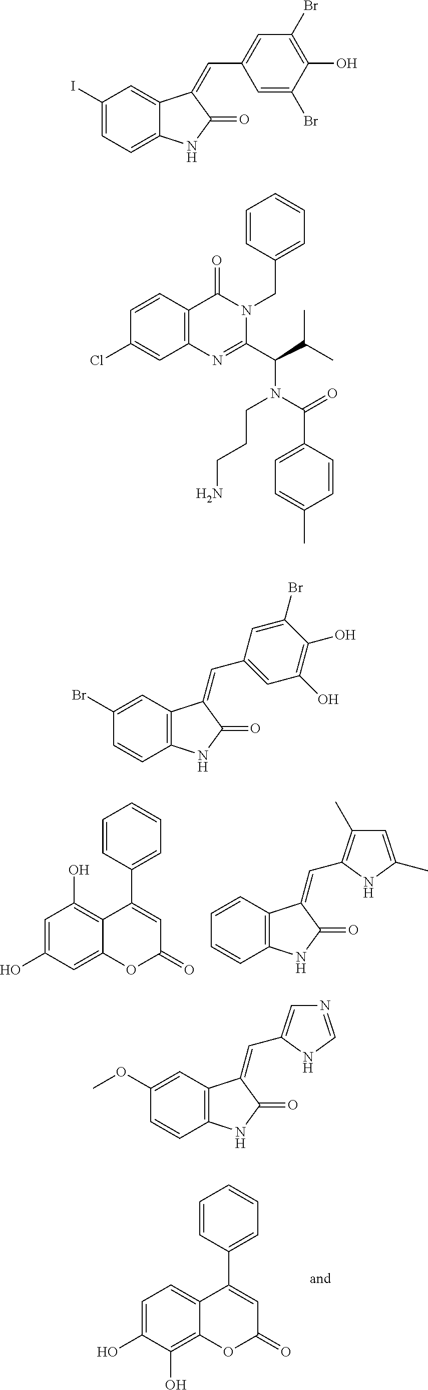

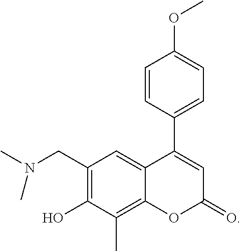 [SMILES: COc1ccc(-c2cc(=O)oc3c(C)c(O)c(CN(C)C)cc23)cc1]

[14] A pharmaceutical composition, detection reagent or kit comprising a compound obtained by the method of any one of [1] to [10] above.

[15] A pharmaceutical composition, detection reagent, kit or screening system, which comprises at least one compound selected from the following:

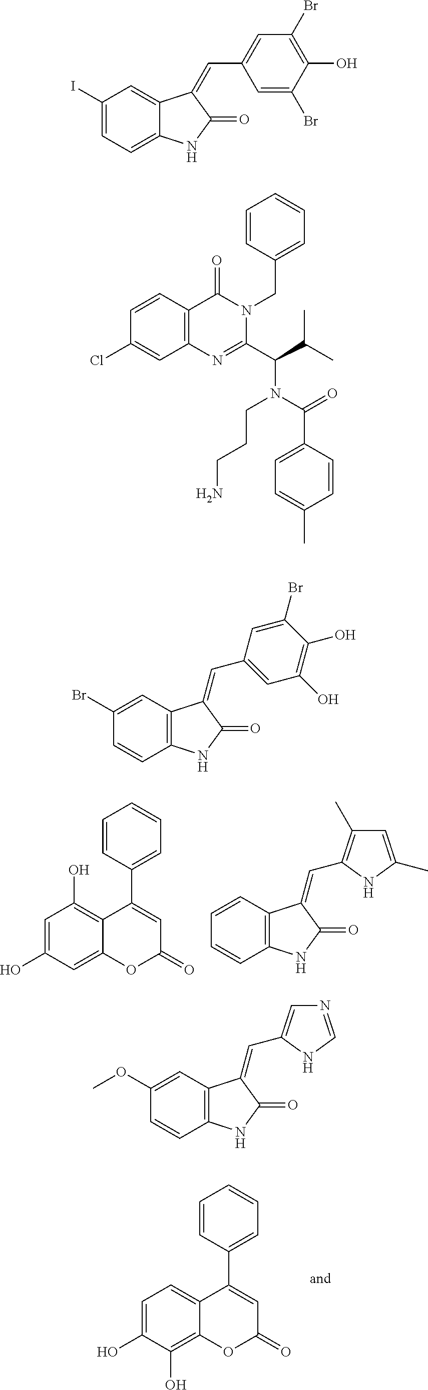

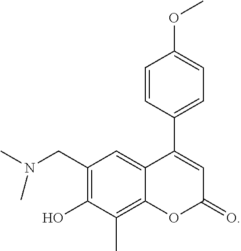 [SMILES: COc1ccc(-c2cc(=O)oc3c(C)c(O)c(CN(C)C)cc23)cc1]

[16] A screening system comprising

(1) a protein with abnormally expanded polyQ or a fragment thereof containing the polyQ part, and

(2) a LC3B protein, a homologue thereof, or a fragment thereof;

preferably, the screening system is in the form of a kit.

[17] Use of a compound or a pharmaceutical composition thereof that positively modulates the binding between (1) a protein with abnormally expanded polyQ or a fragment thereof containing the polyQ part, and (2) a LC3B protein, a homologue thereof, or a fragment thereof in the preparation of a medicament for the treatment or prevention of a polyQ-related neurodegenerative disorder.

### Beneficial Effects

The compound screened by the method of the present invention can selectively reduce the level of the protein with abnormally expanded polyQ in the cell by targeting autophagy, and has no influence on the autophagy function of cells. Therefore, the method of the present invention can be used to effectively screen compounds for treating or preventing polyQ-related neurodegenerative disorders.

Autophagy is a highly genetically conserved protein degradation pathway, which is ubiquitous in eukaryotic cells. It has strong protein degradation ability but shows low selectivity. The existing research mainly focuses on autophagy modulators, which leaves an unsolved problem about the global effect of non-specific regulation of autophagy, and therefore has potential side effects. The inventor unexpectedly discovered that the screening method of the present invention can obtain compounds that specifically and autophagy-dependently reduce the level of proteins with abnormally expanded polyQ, which have low side effects and good safety, and are easy to pass through the BBB, potentially facilitating oral administration. In addition, the screening method of the present invention has the advantages of easy implementation and high screening efficiency.

### EXAMPLES

Unless otherwise specified, the instruments and reagent materials used herein are all commercially available, or can be prepared by conventional methods known in the art.

Unless otherwise specified, in all statistical analyses in this article, * represents p<0.05; ** represents p<0.01; *** represents p<0.001, **** represents p<0.0001. For the comparison between the two groups, the statistical analysis method used is the two-tailed unpaired t test. For comparisons between three or more groups, two-tailed one-way ANOVA was applied when there is only one variable, and two-tailed two-way ANOVA was applied when there are two variables.

### Abbreviation

| | | | |
|---|---|---|---|
| ATXN | ataxin | HTT | Huntingtin |
| TBP | TATA binding protein | ATN | atrophin |
| fl | full length | | |
| BBB | blood-brain barrier | | |
| AR | androgen receptor | | |

| | |
|---|---|
| BDNF | brain derived neurotrophic factor |
| OI-RD | oblique-incidence reflectivity difference |
| MALDI-TOF-MS | matrix-assisted laser desorption/ionization |
| | time of flight mass spectrometry |

### Experimental Materials, Reagents and Method Steps

### Compounds

The compound library used in the examples is provided by Selleck, containing 3375 biologically active compounds. These include 1,527 drugs approved by the U.S. Food and Drug Administration (FDA), 1,053 natural products from traditional Chinese medicine, and 795 known inhibitors. Among them:

Compound 1: GW5074, available from DC Chemicals, catalog number DC8810;

Compound 2: ispinesib, PubChem CID: 6851740, available from Selleck, catalog number S1452;

Compound 3: PubChemID 1759437, available from Specs, catalog number AN-655/15003575;

Compound 4: PubChem CID 5398649, available from ChemDiv, catalog number D715-2435;

Compound 5: Semaxanib, available from Selleck, CAS No. 194413-58-6;

Compound 6: Su9516, available from Selleck, CAS No. 377090-84-1;

Compound 7: available from TargetMol, CAS No. 842-01-3;

Compound 8: available from ChemDiv.

Antibody

HTT antibody 2B7 (Weiss et al. Anal Biochem 2009, 395, 8-15), 4C9, ab1 (Sapp et al. J Biol Chem 2012, 287, 13487-13499) and MW1 (Ko et al. Brain research bulletin 2001, 56, 319-329) were prepared by the method of the prior art; the antibody S830 used for immunostaining to detect HTT aggregates was gifted from Dr. Gillian Bates; other antibodies were purchased from companies such as Millipore and Sigma.

Preparation of Recombinant Human LC3B

(1) His8 tag and TEV protease cleavage site are added to pGEX-6P1 (from GE Healthcare) to prepare prokaryotic expression vector pGHT. LC3B gene (GenBank: NM_022818.4) is edited to remove Met and add two Glys at the N-terminal of LC3B. The gene is amplified and cloned into the prepared pGHT.

(2) The expression plasmid pGHT-LC3B was introduced into E. coli BL21(DE3)pLsyS for expression. Purification is conducted using HisTrap HP column (GE Healthcare, 17524701). Then the protein is mixed with TEV protease (Sigma, T4455) and dialyze overnight. Purification is conducted using HisTrap HP column, Superose 6 Increase 10/300 GL size exclusion column (GE Healthcare) in sequence.

(3) LC3B verification: The prepared human LC3B is verified by MALDI-TOF-MS and X-ray diffraction. The method of X-ray diffraction verification of LC3B is: the mutant LC3BAG120 protein lacking the G120 lipidation site is obtained using site-directed mutagenesis, and this protein with improved stability is used for high-resolution X-ray diffraction. The known LC3 protein crystal structure (PDB ID: 1UGM) is used as a search model for molecular replacement. The published structure of LC3BAG120 (PDB ID: 6J04, 1.90 Å) was solved by molecular replacement and structural alignment was performed.

Preparation of Recombinant Human HTTexon1-MBP Protein

(1) 25Q or 72Q HTTexon1 cDNA is synthesized de novo, cloned into mammalian expression vector pTT5SH8Q2. A C-terminal MBP tag is added after the HTTexon1 sequence to generate the pTT-HTTexon1-Q72-MBP and pTT-HTTexon1-Q25-MBP plasmids.

(2) The plasmid is transfected into HEK293T cells for expression with linear polyethyleneimine (Polysciences, 24765). The protein is purified using HisTrap HP column and Superose 6 Increase 10/300 GL size exclusion column.

(3) The purified HTTexon1-MBP protein was dialyzed into 5 mM NH₄Ac through the Superose 6 Increase size exclusion column, verified by Bruker FLEX MALDI-TOF.

Preparation of Recombinant Human Full-Length HTT Protein

(1) The human HTT gene (GenBank: NM_002111.8) with (CAG)₂₃ or (CAG)₇₃ was synthesized de novo by Genewiz Inc. The human HTT gene was cloned into the modified pCAG vector (from Addgene) with N-terminal protein A tag.

(2) The plasmid was transfected into human embryonic kidney E293 cells for expression using polyethyleneimine (PEI, from Polysciences, 23966). The protein is purified with IgG monoclonal antibody-agarose (Smart-lifesciences, SA030010), digested with TEV protease to remove the protein A tag, and further purified with Mono Q and Superose 6 (5/150 GL) columns from GE healthcare. Validation is conducted using Coomassie blue staining and Western-blots.

Preparation of Recombinant Human MBP-ATXN3

(1) His8 tag and TEV protease cleavage site is added in pMal-C2x plasmid (from New England Biolabs) to prepare prokaryotic expression vector pMBP. The ATXN3 gene (GenBank: NM_001127696.2) is edited to control the length of the expanded polyglutamine, the gene is amplified and cloned into the prepared pMBP to obtain the pMBP-ATXN3-Q28 and pMBP-ATXN3-Q68 plasmids with polyQ repeated 28 and 68 times, respectively.

(2) The expression plasmids pMBP-ATXN3-Q28 and pMBP-ATXN3-Q68 were introduced into Escherichia coli Rosetta(DE3)pLsyS for expression. Preliminary purification was conducted with HisTrap HP column (GE Healthcare, 17524701). The product was concentrated by ultrafiltration and then further purified with Superose 6 Increase 10/300 GL size exclusion column (GE Healthcare).

(3) As verified by SDS-PAGE, the purity of the prepared human MBP-ATXN3-Q28 and MBP-ATXN3-Q68 exceeds 98%.

Cells Used for Tests

Primary cultured cortical neurons: The brains of Hdh^{Q140/Q7} and Hdh^{Q7/Q7} newborn mice (P0) were dissected, digested, dissociated, and cultured.

Some primary patient fibroblasts and wild-type cells are from HD patients (Q47, Q55) of the Mongolian Huntington's disease family and healthy controls (WT, Q19). SCA3 cell line was from patient (Q74). The HD Q68 fibroblast cell line was from Coriell Cell Repositories (Camden, N.J., USA). Immortalized fibroblasts and induced stem cells (iPSCs) are prepared from primary fibroblasts. Mouse striatum cells (STHdh) were from Coriell Cell Repositories (Camden, N.J., USA). HEK293T cells and HeLa cells were from ATCC.

Animals Used for Tests

Huntington's Disease Drosophila

Nervous system driver line elav-GAL4 (c155), HTT expressing lines UAS-fl-HTT-Q16 and UAS-fl-HTT-Q128 were from Bloomington Drosophila Stock Center (http://flystocks.bio.indiana.edu/) and kept in a 25° C. incubator.

Crosses were set up between virgin female Drosophilae carrying elav-GAL4 driver and the UAS-fl-HTT-Q16 or UAS-fl-HTT-Q128 male Drosophilae to generate a transgenic Drosophilae driven by elav-GAL4 to express human HTT full-length protein (Q16) or (Q128) in the nervous system.

Huntington's Disease Mice

Mice expressing wild-type HTT gene (Hdh^{Q7/Q7}) were from the Marian Difiglia laboratory of Massachusetts General Hospital, Harvard University. The Q140 gene knock-in heterozygous mouse (Hdh^{Q140/Q7}) was prepared according to the method of the prior art (Menalled et al., J Comp Neurol, 2003, 465: 11-26).

Protein Analysis

Homogeneous time-resolved fluorescence (HTRF) analysis: the cell or tissue lysate was diluted with the original lysis buffer PBS+1% (v/v) Triton X-100+1×cOmplete™ protease inhibitor to lyse the sample, then HTRF assay buffer (50 mM NaH₂PO₄, 400 mM NaF, 0.1% BSA, 0.05% (v/v) Tween-20, 1% (v/v) Triton X-100, pH 7.4) was used to dilute the specified antibody pair for detection. In HTRF buffer, the donor antibody concentration was 0.023 ng/μL and the acceptor antibody concentration was 1.4 ng/μL.

Determination of the amount of protein: the amount of protein was determined by the above method. Background correction was performed using blank sample. The protein concentration for all samples were determined to correct the loadings. Different protein concentrations or cell numbers per well were measured to ensure that the signals are within the linear range.

Cell Analysis

Immunofluorescence: cells were washed, fixed, permeabilized and blocked, then incubated with primary antibody overnight at 4° C., then washed three times with blocking buffer, and incubated with secondary antibody for 1 hour at room temperature, stained with DAPI, and mounted. Images were taken by Zeiss Axio Vert A1 confocal microscope. TUBB3 or co-localization was analyzed using ImageJ.

### Example 1 High-Throughput Screening of Compounds for Protein Affinity

### 1.1 Affinity Screening

(1) Compound chips were prepared using a contact microarray printer (SmartArrayer 136, CapitalBio Corporation) according to the prior art (Zhu et al., Sensors (Basel) 2016, 16(3), 378; Fei et al., J Biomed Opt 2010, 15(1), 016018). Each compound was printed in duplicate on the microarray. The compound not fixed on the surface of the chip was washed away. The chip was sealed and cleaned. 01-RD microscope was applied to scan the images. HTTexon1-Q72-MBP (SEQ ID NO: 1 with MBP tag added, wherein MBP tag was used to increase solubility) was used as the target protein. The compound chip was incubated with the target protein (at a concentration of 238 nM) for 2 hours. The chips are cleaned and scanned.

(2) HTTexon1-Q25-MBP (SEQ ID NO: 5 with MBP tag added) was used as the target protein (concentration 454 nM). OI-RD images before and after incubation with the target protein was obtained following the method in (1).

(3) LC3B (SEQ ID NO: 8) was used as the target protein (concentration 680 nM). 01-RD images before and after incubation with the target protein was obtained following the method in (1).

The difference images of the above steps (1), (2), (3) (the image after incubation minus the image before incubation) was analyzed respectively. Compounds that bind to HTTexon1-Q72-MBP and LC3B but not to HTTexon1-Q25-MBP (the detection limit of 01-RD microscope was about 0.001 mV) were selected. The following compounds which can bind to LC3B and HTTexon1 with abnormally expanded polyQ, but do not bind to HTTexon1 with normal polyQ were obtained: Compound 1 (GW5074) and compound 2 (ispinesib).

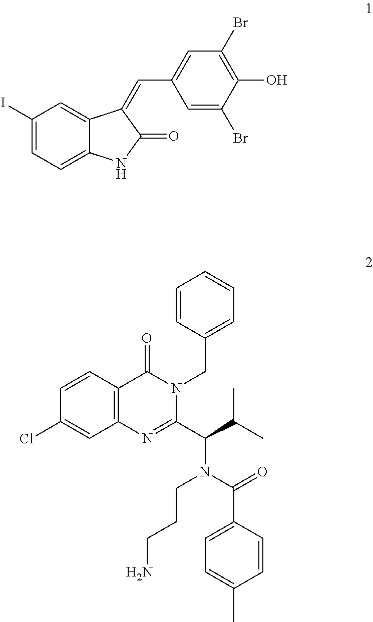 [SMILES: O=C1Nc2ccc(I)cc2/C1=C/c1cc(Br)c(O)c(Br)c1 ; Cc1ccc(C(=O)N(CCCN)[C@@H](c2nc3cc(Cl)ccc3c(=O)n2Cc2ccccc2)C(C)C)cc1]

Chips with compounds 1 and 2 are prepared according to the above method. Each compound was printed in triplicates on the microarray. 01-RD was used to measure the affinity reaction parameters of compounds with HTTexon1-Q72 and HTTexon1-Q25.

The compounds showed no binding affinity with HTTexon1-Q25. The affinity reaction parameters with HTTexon1-Q72 are shown in Table 1.

TABLE 1
The association rate constant [symbol] dissociation rate constant [symbol]
and equilibrium dissociation constants of the affinity
reactions between the compounds with HTTexon1-Q72

| | Kₒₙ (min•nM)⁻¹ | K_{off} (min)⁻¹ | K_{d} (nM) |
|---|---|---|---|
| compound 1 | 9.07 × 10⁻⁵ | 1.43 × 10⁻² | 157.6 |
| compound 2 | 2.13 × 10⁻⁴ | 6.23 × 10⁻³ | 29.2 |

Not more than 20 compounds were selected from commercially available known compounds which had the following: bicyclic structures and side chains containing benzene rings like in compounds 1 and 2, and reactive functional groups (such as OH, SH, NH, etc.) that are required for preparing small molecule chips. Screening was conducted according to the above-described method, and two compounds that can bind to HTTexon1-Q72 and LC3B but not to HTTexon1-Q25 were obtained: compound 3 and compound 4.

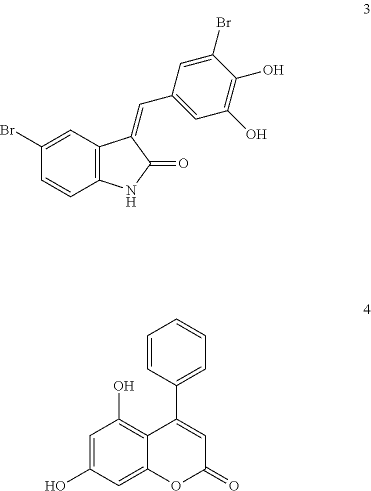 [SMILES: O=C1Nc2ccc(Br)cc2/C1=C/c1cc(O)c(O)c(Br)c1 ; O=c1cc(-c2ccccc2)c2c(O)cc(O)cc2o1]

Chips with compounds 1, 2, 4 were prepared according to the above method. Each compound was printed in triplicates on the microarray. 01-RD was used to measure the affinity reaction parameters of the compound with LC3B, full-length mHTT (flHTT-Q73) and full-length HTT-Q23 (flHTT-Q23).

The compound showed no binding affinity with flHTT-Q23 (SEQ ID NO: 4). The affinity reaction parameters with flHTT-Q73 and LC3B are shown in Table 2 and Table 3, respectively.

TABLE 2
The association [symbol] rate constants, dissociation rate
constants, and equilibrium dissociation constants of the
affinity reactions between the compounds and flHTT-Q73

| | Kₒₙ (min•nM)⁻¹ | K_{off} (min)⁻¹ | K_{d} (nM) |
|---|---|---|---|
| compound 1 | 5.43 × 10⁻⁵ | 7.97 × 10⁻³ | 14.7 |
| compound 2 | 6.43 × 10⁻⁵ | 2.07 × 10⁻² | 321.9 |
| compound 4 | 4.81 × 10⁻⁴ | 1.19 × 10⁻² | 24.7 |

TABLE 3
The association rate constant [symbol] dissociation rate
constant [symbol] and equilibrium dissociation constants of the
affinity reactions between the compounds and LC3B

| | Kₒₙ (min•nM)⁻¹ | K_{off} (min)⁻¹ | K_{d} (nM) |
|---|---|---|---|
| compound 1 | 3.72 × 10⁻⁵ | 1.74 × 10⁻² | 467.7 |
| compound 2 | 2.46 × 10⁻⁵ | 5.86 × 10⁻³ | 238.2 |
| compound 4 | 4.61 × 10⁻⁵ | 2.49 × 10⁻² | 540.1 |

1.2 MST Detection of the Affinity of the Compound with Full-Length HTT and LC3B

The affinity of the compound with full-length HTT and LC3B was verified with micro thermophoresis (MST) using Monolith NT.115 instrument from NanoTemper Technologies. The reaction buffer was 20 mM HEPES, pH 7.4, 150 mM NaCl. Protein concentration was 500 nM. The compound showed no binding affinity with flHTT-Q23 (SEQ ID NO: 4), and the K_{d} of flHTT-Q73 and LC3B are shown in Table 4, respectively.

TABLE 4
The affinity reaction equilibrium dissociation
constant between the compounds and the test protein

| K_{d} (μM) | compound 1 | compound 2 | compound 3 | compound 4 |
|---|---|---|---|---|
| flHTT-Q73 | 3.46 | 0.13 | 1.00 | 2.82 |
| LC3B | 4.76 | 3.10 | 2.35 | 3.95 |

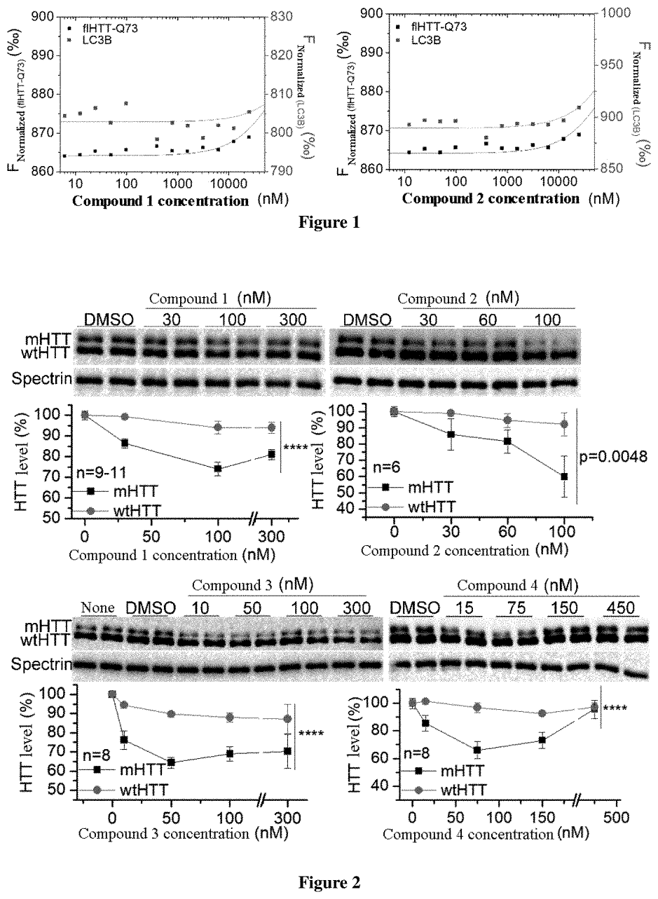

The inventors also verified through MST that the compound had respective affinity with both full-length HTT and LC3B in a system where both full-length HTT and LC3B existed (FIG. 1).

In summary, the results of 01-RD measurement and MST assay both showed that the compound had binding affinity to LC3B; for HTT, the compound selectively binded mHTT (HTTexon1-Q72 or flHTT-Q73). This indicated that the compound can positively regulate the binding effect between LC3B and mHTT.

### Example 2 Effects of Compounds on Cortical Neurons in HD Model Mice

2.1 Effects on mHTT and wtHTT Levels

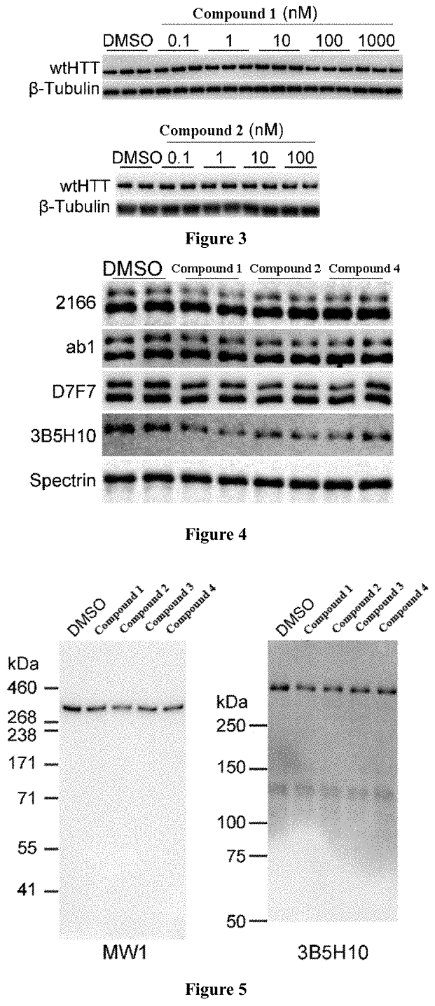

(1) The primary cultured Hdh^{Q140/Q7} and Hdh^{Q7/Q7} mouse cortical neuron cells were treated with the compound for 2 days. Then the levels of mHTT and wtHTT were detected by Western-blots (antibody 2166) (FIG. 2 and FIG. 3). The compound reduced the level of mHTT in cortical neurons of Q140 knock-in heterozygous mice (Hdh^{Q140/Q7}), but hardly affected the level of wtHTT. Compounds 1 and 2 did not reduce wtHTT levels in cortical neurons of Hdh^{Q7/Q7} mice. When compound 1 was in the range of 30-300 nM (100 nM was preferred), compound 2 was in the range of 30-100 nM (100 nM was preferred), compound 3 was in the range of 10-300 nM (50 nM was preferred), compound 4 was in the range of 15-150 nM (75 nM was preferred), the effect of selectively reducing mHTT levels was observed.

(2) Using a test method similar to (1), the levels of mHTT in Hdh^{Q140/Q7} mouse cortical neurons after compound treatment were detected using a variety of antibodies (2166, ab1, D7F7, 3B5H10) (FIG. 4). The variety of antibody tests gave relatively consistent results, so the detected decrease in mHTT level was not due to changes in the affinity of mHTT for specific antibodies.

mHTT was detected using anti-polyQ antibodies MW1 and 3B5H10, and observed for bands of proteins with smaller molecular weights. As a result, no increase in the N-terminal fragments of mHTT was observed (FIG. 5). The detected decrease in mHTT level was not due to an increase in site-specific cleavage.

### 2.2 Cytotoxicity Test

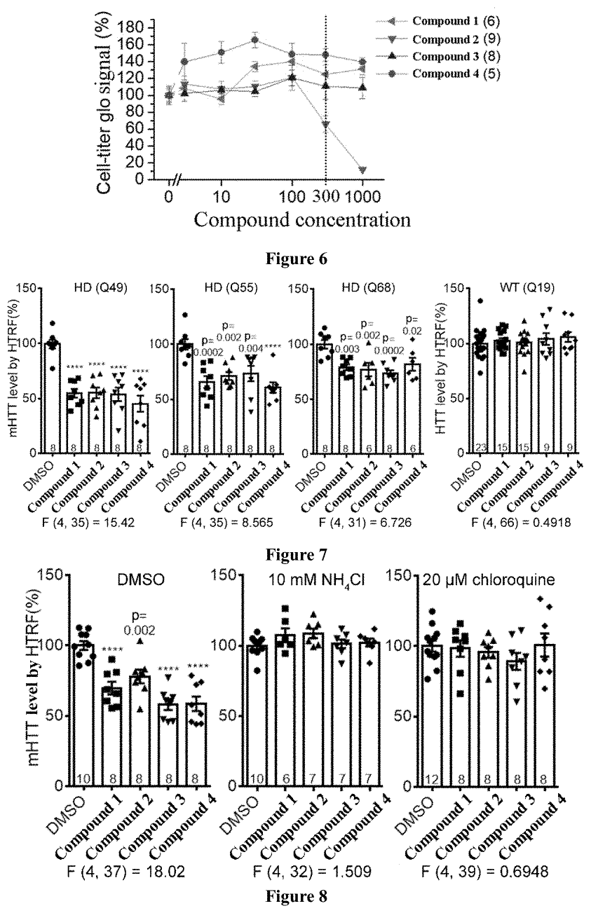

CellTiter-glo (Promega, G7570) was used according to the protocol provided in the kit to determine the viability of Hdh^{Q140/Q7} mouse cortical neurons after compound treatment (FIG. 6). The compound showed no cytotoxicity to Hdh^{Q140/Q7} mouse cortical neurons at the test concentration described in 2.1. The detected decrease in mHTT level was not due to neuronal cell loss.

In summary, the compounds obtained by the screening method of the present invention all exhibited excellent allele-selective effects in reducing mHTT levels in cells. Among them, two compounds that was verified to be active at the cellular level were obtained from a structurally diverse compound library containing 3375 biologically active compounds, demonstrating a positive rate of 0.6‰.

### Example 3 Effects of Compounds on HTT Level in Fibroblasts of Huntington's Disease Patients

3.1 Effect on the Levels of mHTT and wtHTT in Primary Fibroblasts of HD Patients

Preliminary experiments were conducted. As a result, the compound exhibited the best mHTT reduction effect at a concentration of 100 nM.

Experiment method: The fibroblasts (Q49, Q55, Q68) of HD patients were treated with 100 nM compound for 2 days. Then mHTT (antibody pair: 2B7/MW1) and total HTT (antibody pair: 2B7/2166) were detected by HTRF. The results are shown in FIG. 7. Reduced mHTT levels were observed in primary fibroblasts (Q49, Q55, Q68) of HD patients. No reduction in HTT levels was observed in wild-type primary fibroblasts.

3.2 Effects on the Level of mHTT in HD Patient Immortalized Fibroblasts

A test method similar to that described in 3.1, HTRF (antibody pair: 2B7/MW1) was used to detect the effects of the compound on the level of mHTT in immortalized fibroblasts of HD patients in the presence or absence of autophagy inhibitors (FIG. 8). In the absence of autophagy inhibitors, the compound significantly reduced the mHTT level of HD patient immortalized fibroblasts, which is consistent with the results observed on HD patient primary fibroblasts. On the contrary, in the presence of autophagy inhibitors (NH₄Cl or chloroquine), no decrease in mHTT levels was observed.

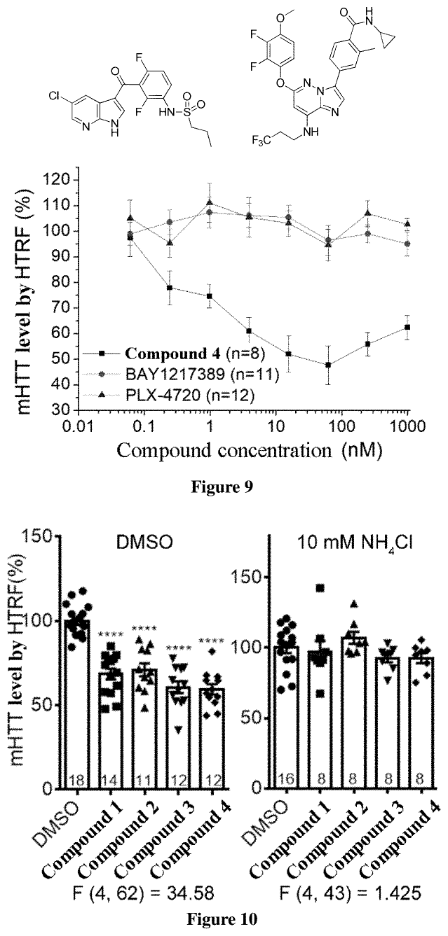

It was reported that compound 1 also had c-Raf inhibitor activity, and compound 2 also had kinesin spindle kinase (KSP) inhibitory activity. For this reason, the inventors tested some c-Raf inhibitors and KSP inhibitors (including GSK923295, BAY1217389, PLX-4720, Dabrafenib, Sorafenib Tosylate). The immortalized fibroblasts of HD patients were treated using the above-mentioned c-Raf inhibitors and KSP inhibitors respectively at a concentration of 100 nM. No decrease in mHTT level was observed. Using PLX-4720 (c-Raf inhibitor), BAY1217389 (KSP inhibitor) and compound 4 for comparison, PLX-4720 and BAY1217389 did not reduce mHTT levels at concentrations below the micromolar level (FIG. 9). Therefore, the effect of the compound on mHTT was not caused by the inhibitory activity on c-Raf or KSP.

In summary, the reduction of mHTT levels by the compounds obtained from screening were autophagy-dependent.

### Example 4 Effects of the Compounds on mHTT Level and Neuronal Apoptosis of HD Patient iPSC-Derived Neurons

4.1 Effect on mHTT and wtHTT levels

A test method similar to that described in 3.1, HTRF (antibody pair: 2B7/MW1) was used to detect the effects of compound 1, compound 2, compound 3, and compound 4 on the mHTT level of HD patient iPSC derived neurons (Q47) (FIG. 10). Decreased mHTT levels were observed in HD patient iPSC-derived neurons, and this effect was not observed in the presence of the autophagy inhibitor NH₄Cl.

Using the same method as above, the following compound 5, compound 6, compound 7, and compound 8 were obtained from screening. These compounds were observed to reduce the mHTT level in the HD patient iPSC-derived neurons; wherein compound 5 reduced mHTT levels by approximately 12.2%, compound 6 reduced mHTT levels by approximately 24.9%, compound 7 reduced mHTT levels by approximately 24.2%, and compound 8 reduced mHTT levels by approximately 19.7%. The compound rescued disease-related phenotypes in HD patient iPSC-derived neurons through autophagy.

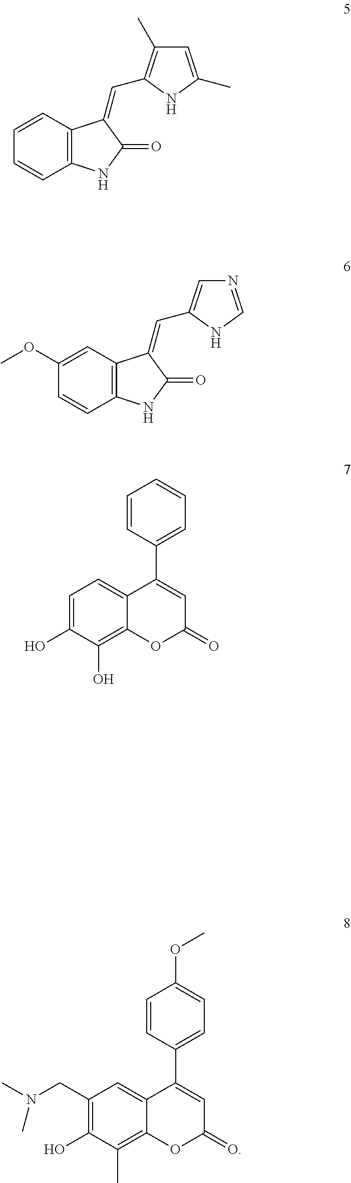

### 4.2 Effects on Neuronal Apoptosis

(1) Immunostaining

HD patient iPSC-derived neurons (Q47) were treated using 100 nM compound 1, or 100 nM compound 2, or 100 nM compound 3 or 50 nM compound 4 for 1 day. Then the cells were stressed (by BDNF removal).

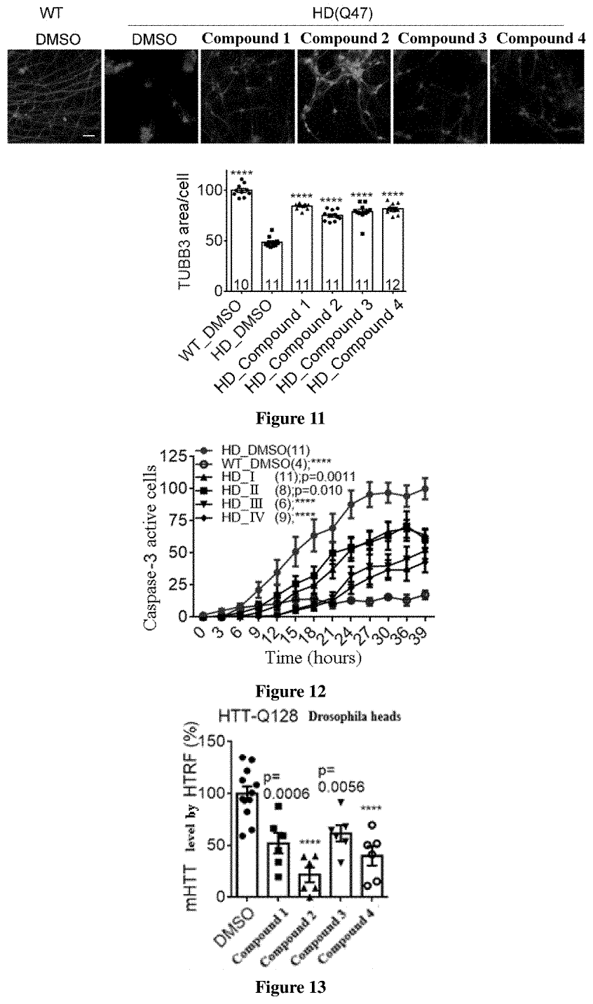

Neuronal specific tubulin marker TUBB3 was stained with DAPI. The TUBB3 signal covered area was normalized to the cell nuclei counts, and the data was normalized to the wild type controls to analyze neuronal apoptosis (FIG. 11).

(2) Caspase-3 Activity Detection

After removing BDNF, loss of processes and shrinkage of neurons were observed in HD neurons.

NucView 488 (Biotium, 30029) was used to detect active caspase-3. After removing BDNF, Incucyte (Essen Bioscience, IncuCyte FLR) was used to capture images every 3 hours in the incubator, which were analyze with Incucyte 2011A software. Three batches were tested and the results were consistent (FIG. 12). The compound significantly improved the loss of processes and shrinkage of HD neurons after removal of BDNF.

### Example 5 Effects of Compounds on Huntington's Disease Drosophilae

5.1 Effects on mHTT Level

Experimental method: Q128 Drosophilae and Q16 Drosophilae were randomized into a negative control group and a positive drug group (compound 1, compound 2, compound 3, compound 4), with 75 drosophilae in each group. The negative control group was given the corresponding solvent DMSO, and the positive drug groups was given the corresponding positive drug.

Drosophilae were kept in standard food at 25° C. The newly hatched Drosophilae were transferred to vials with food containing the positive drug (10 μM in 400 DMSO) or DMSO for control. The food was changed every other day.

After fed for 6 consecutive days, the Drosophila head protein was extracted on the 7th day. The mHTT level was measured by HTRF (antibody pair: 2B7/MW1), where each sample included head proteins from five Drosophila (FIG. 13). The compound reduced mHTT levels in transgenic Drosophilae expressing human HTT full-length protein (Q128).

### 5.2 Effects of Compound on Survival Rate

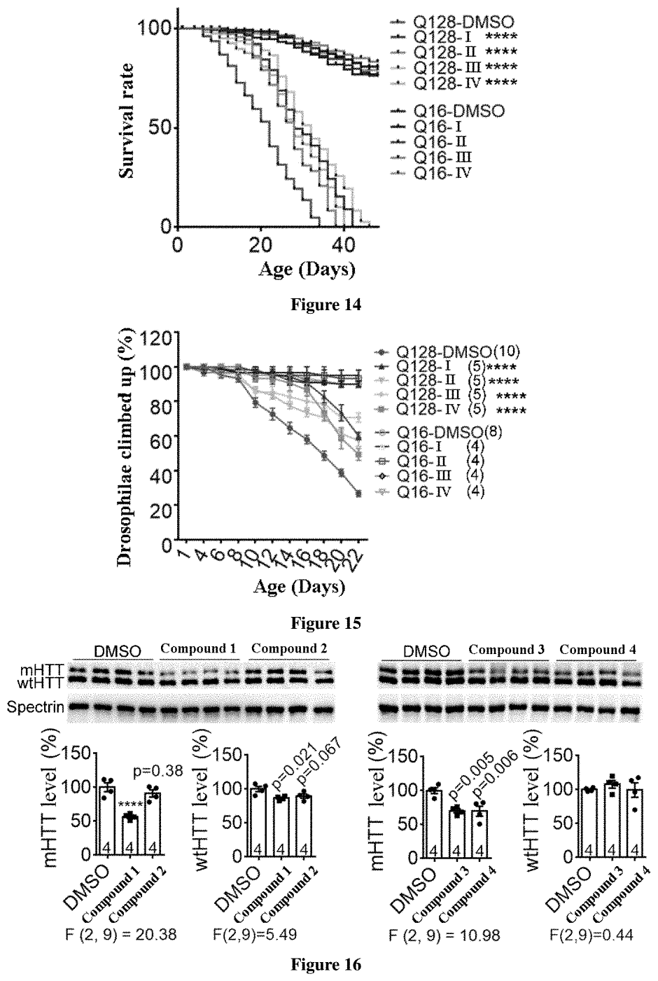

75 age-matched virgin Drosophilae were put into empty plastic vials containing standard food, and the survival rate of each vial was recorded daily to measure the lifespan (FIG. 14). The survival rate of the Q128 Drosophila positive drug group was improved compared to the control group.

### 5.3 Effects on Climbing Performance

15 age-matched virgin Drosophilae was put into empty vials and tapped down so that they were at the bottom of the vials. The percentage of Drosophilae that had climbed past a 7-cm-high line after 15 seconds was record. The mean of five observations for each vial was plotted every day, and the data from multiple vials containing different batches of Drosophilae was plotted and analyzed (FIG. 15). The number in brackets indicates the number of tested vials. The Q128 Drosophila positive drug group showed improved the climbing performance compared to the control group.

In the above experiments in 5.2 and 5.3, no compound was observed to have an effect on Q16 Drosophilae.

### Example 6 Effects of Compounds on HD Model Mice

The mice were grouped and housed in individually vented cages with a 12-hour light/dark cycle, with a maximum of 5 adult mice per cage.

6.1 Effects of Intracerebroventricular Injection of Compounds on the Levels of mHTT and wtHTT in the Cortices of HD Mice

Experimental animals: Hdh^{Q140/Q7} mice (3 months old), 4 in each group.

Experimental method: one icv-injection was conducted per day using 2 μL artificial cerebrospinal fluid (ACSF: 1 mM glucose, 119 mM NaCl, 2.5 mM KCl, 1.3 mM MgSO₄, 2.5 mM CaCl₂), 26.2 mM, NaHCO₃, 1 mM NaH₂PO₄) containing a compound at a concentration of 25 μM. 2 μL ACSF containing the same amount of DMSO was used as a control.

After 10 days of injection, the brain cortical neuron proteins of the mice were extracted, and the levels of mHTT and wtHTT were detected by Western-blots (antibody 2166). Each test includes three repetitions for each sample, and the mean values was calculated (FIG. 16). Compounds 1, 3 and 4 significantly reduced mHTT level in Hdh^{Q140/Q7} mouse cortices and showed mHTT selectivity relative to wtHTT.

### 6.2 Compound 1 or 4 Administered by Intraperitoneal Injection

Experimental method: The compound or control DMSO was diluted with 0.9% NaCl intravenous infusion solution to 0.05 μg/μL. One ip-injection (0.5 mg/kg) was conducted per day. After 14 days of injection, tissue extraction or behavioral experiment were conducted.

In vivo compound detection in brain tissues of ip-injected mice: 2 hours after ip-injection of DMSO or compound (compound 1 or compound 4) in mice, the brain was dissected, weighed, and the compound in the brain tissue was extracted, analyzed with UPLC-MS (Acquity Ultra Performance Liquid Chromatography System, Acquity UPLC BEH C18 (1.7 μm, 2.1×50 mm) column and Xevo TQ-S mass spectrometer, Waters Corporation, Milford, Mass., USA) for LC-MS/MS analysis. The results showed that the level of compound 1 reached the brain tissue was 81.0 ng/g. The level of compound 4 reached the brain tissue was 9.48 ng/g. No mass spectrum signal of compound was observed for the control (DMSO) group. The compound can be delivered to the brain of mice through the BBB smoothly, which potentially facilitates oral administration of the compound.

6.3 Effects of Intraperitoneal Injection of Compounds on the Levels of mHTT and wtHTT in the Cortices and Striata of HD Mice

(1) 20 Hdh^{Q140/Q7} mice (5 months old) were randomized into 3 groups.

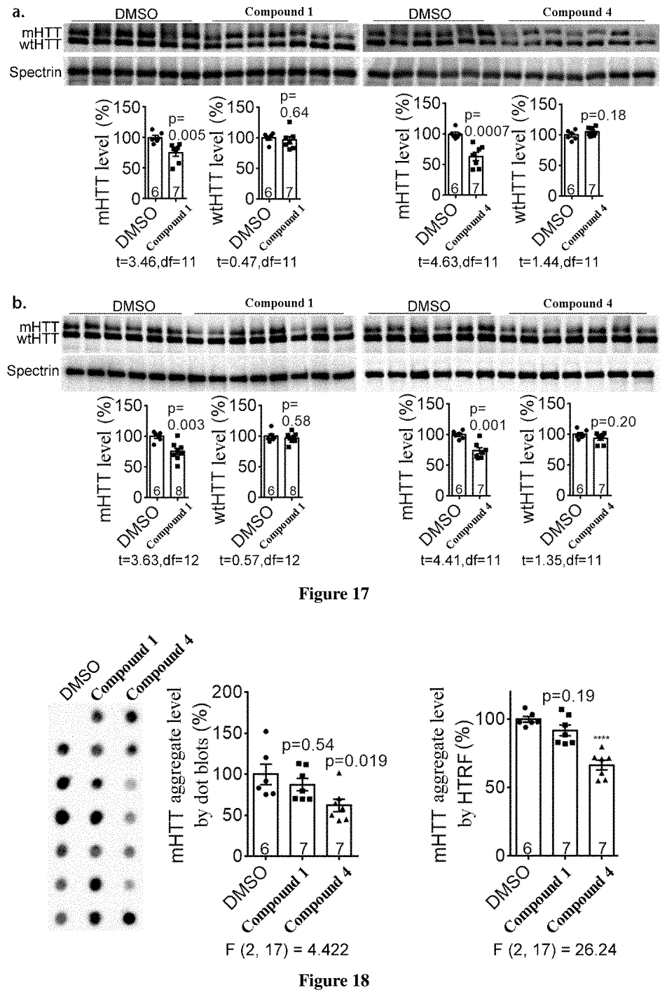

Administer compound 1 or 4 as described in 6.2. One ip-injection was conducted per day. After 14 days of injection, the protein was extracted and the levels of mHTT and wtHTT in the cortices of HD mice were detected by Western-blots (FIG. 17a).

(2) A total of 21 mice Hdh^{Q140/Q7} mice (10 months old) were used, 6-8 mice in each group.

According to the above method, the mouse brain striatum neuronal protein was obtained and the levels of mHTT and wtHTT were detected by Western-blots (FIG. 17b).

(3) mHTT aggregates in the cortices of Hdh^{Q140/Q7} mice were detected by dot-blot experiment (antibody: 4C9, bar plot showed the results of two repetitions) and HTRF (antibody pair: 4C9/4C9). Sampling was repeated for each mouse two to three times on average (FIG. 18). No increase in mHTT aggregates was observed. The decrease in mHTT levels in the compound-treated group was not due to changes in mHTT solubility.

In summary, intraperitoneal injection of the compound reduced the levels of mHTT in the cortices and striata of Hdh^{Q140/Q7} mice, and showed mHTT selectivity relative to wtHTT. Therefore, the compound had the prospect of being developed to an oral drug.

### 6.4 Effects of Intraperitoneal Injection of Compounds on Behavioral Defects in HD Mice

Experimental animals: 42 Hdh^{Q140/Q7} mice were randomized into 3 groups; 48 Hdh^{Q7/Q7} mice were randomized into 3 groups.

Experimental method: administer compound 1 or 4 according to the procedure described in 6.2. One ip-injection was conducted per day, and behavioral experiments were conducted after 14 days of injection.

All behavioral experiments were carried out during the light phase. Before starting the experiments, all mice were kept in the behavioral test room under dim red light for one hour.

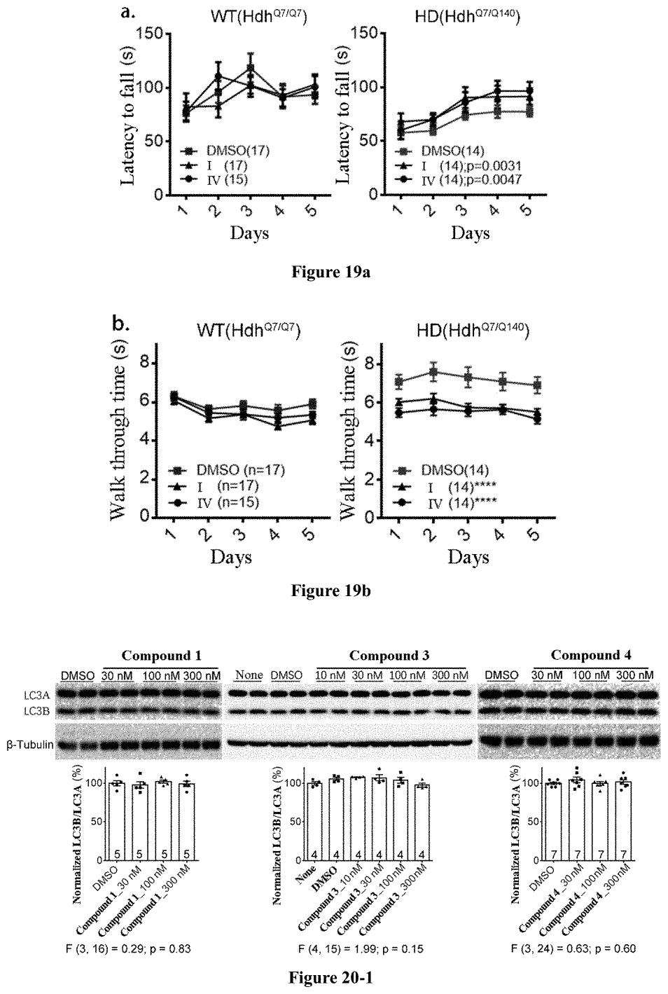

Rotarod test: The mice were pre-trained for 3 consecutive days (on the rotarod rotating at 4 rpm for 2 minutes). The result of each experiment was recorded as time on the rod (time on the rotating rod), until falling from the rod or until the end of the task. Each test include three repetitions with an inter-trial interval of 60 min in order to reduce stress and fatigue. The means of three trials were analyzed for each mouse (FIG. 19a).

Balance beam test: a 2 cm thick meter stick with a total length of 100 cm was suspended on the platform on both sides. There was a bright light at the starting point and a dark box with food at the endpoint. The total time for each mouse to walk through the balance beam was recorded (FIG. 19b).

In the rotating rod test, F (2, 195)=4.963; in the balanced beam test, F (2, 195)=37.31. The compound of the present invention improved the Huntington's disease related behavioral defects in HD model mice, and had no effect on wild-type mice.

### Example 7 Compounds do not Affect Autophagy Function and the Level of Control Proteins

### 7.1 Effects of Compounds on Cultured Hdh^{Q140/Q7} Mouse Cortical Neurons

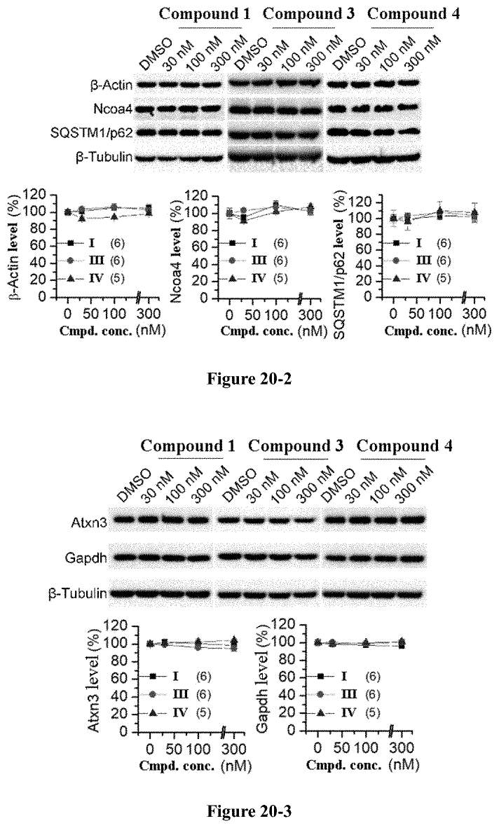

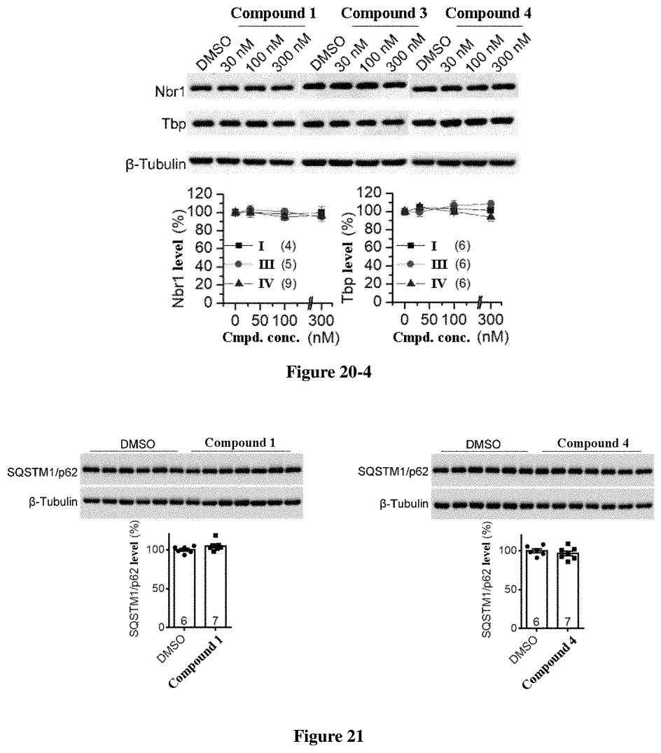

Following the method similar to 2.1, the level of LC3B, other known autophagy selective substrate proteins (including SQSTM1/p62, NBR1 and Ncoa4), other polyQ proteins with wild-type length polyQ (Atxn3 and Tbp) and several control housekeeping proteins in the cortical neurons of Hdh^{Q140/Q7} mice after compound treatment were detected. The results of Western blotting are shown in FIG. 20-1, FIG. 20-2, FIG. 20-3, FIG. 20-4. The compound did not affect the expression level of LC3B in the primary cultured cortical neurons, and the levels of other proteins tested were not affected by the compound (protein level change<10%).

### 7.2 Test the Effects of Intraperitoneal Injection of Compounds

Using a method similar to 6.2, Hdh^{Q140/Q7} mice were administered with injections. A method similar to 7.1 was used to detect the effects of ip-injection of compounds on other known autophagy selective substrate proteins (SQSTM1/p62) in the cortical tissue of Hdh^{Q140/Q7} mice. The result of Western blotting is shown in FIG. 21. After ip-injection of compound 1 or compound 4, the level of SQSTM1/p62 in the cortices of HD mice did not change.

In summary, the detected reduction in mHTT was not due to changed autophagy functions.

### Example 8 Effects of Compounds on the Binding Between mHTT and LC3B

### 8.1 Effects of Compounds on the Binding Between HTTexon1 and LC3B

Protein binding experiment in vitro (in vitro pull-down assay)

(1) The purified recombinant human HTTexon1-MBP (SEQ ID NO: 1 or SEQ ID NO: 5 with MBP tag added) and MBP used for control were respectively immobilized on amylose resin (New England BioLabs, E8021L) through affinity.

(2) The resin obtained in (1) containing about 10 g MBP-fusion protein or MBP was incubated with the compound (1 μM compound 1 or 100 nM compound 4) or the corresponding solvent DMSO, respectively. Then 40 μg of purified LC3B (corresponding to SEQ ID NO: 8) was added. Incubation was conducted. Then the unbound proteins were removed.

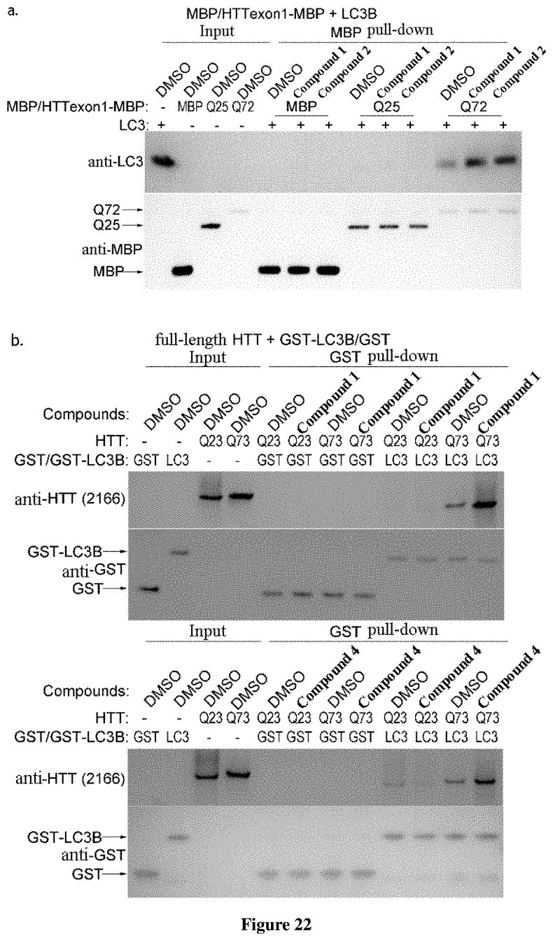

(3) The resin-bound proteins were eluted and analyzed by SDS-PAGE and Western-blots with anti-LC3 antibody (FIG. 22a). Compound 1 and Compound 4 could enhance the binding between HTTexon1-Q72-MBP and LC3B, but not between HTTexon1-Q25-MBP and LC3B.

8.2 Effects of Compounds on the Binding Between flHTT-Q73 and LC3B

GST-LC3B and GST for control were immobilized on mouse anti-GST antibody coupled magnetic beads (Cell Signaling Technology, 11847S). Pulled-down experiment was conducted using a method similar to 8.1. The GST-LC3B or GST were pulled down using full-length HTT (flHTT-Q73 or flHTT-Q23), respectively (FIG. 22b). The pull-down loading ratio was: 100% for GST-LC3B or GST, 10% for full-length HTT. Elution was conducted with SDS-PAGE protein loading buffer. The compound enhanced the binding between full-length mHTT and LC3B.

### Example 9 Effects of Compounds on the Localization of mHTT and LC3B in Cells

### 9.1 Co-Localization Between HTTexon1 and LC3B in Hela Cells Under the Influence of Compounds

(1) The HTTexon1 cDNA was subcloned into the mammalian expression vector pTT-MBP-His to prepare pTT-HTTexon1-Q72-MBP-His and pTT-HTTexon1-Q25-MBP-His.

(2) The pTT-HTTexon1-Q72-MBP-His and pTT-HTTexon1-Q25-MBP-His prepared in (1) and pEX-GFP-hLC3WT (Addgene, 24987) were transiently transfected into HeLa cells to express HTTexon1-MBP-His (SEQ ID NO: 1 with MBP-His tag added and SEQ ID NO: 5 with MBP-His tag added) and LC3B-GFP (based on SEQ ID NO: 8).

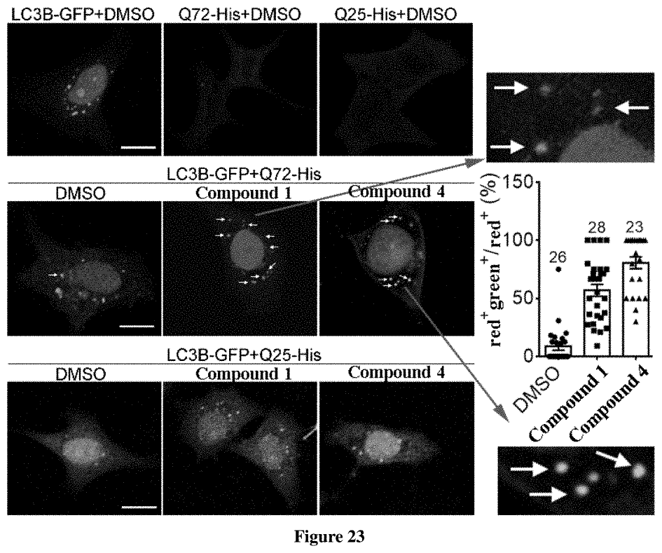

(3) The cells prepared in (1) or (2) were treated with 100 nM compound 1 or 50 nM compound 4, and the cells were mounted 4 hours later. Zeiss Axio Vert A1 confocal microscope was used to detect HTTexon1-MBP-His by anti-His immunofluorescence and LC3B-GFP by GFP fluorescence. The fluorescence signal of the former was red, and the fluorescence signal of the latter was green. Cells transfected with LC3B-GFP alone were detected in the His channel, and cells transfected with HTTexon1-MBP-His alone were detected in the GFP channel to verify the specificity of the detection method. The puncta imaged at both channels were counted and the co-localization was analyzed (FIG. 23). The y-axis of the histogram in FIG. 23 showed the proportion of the red⁺ (HTTexon1-MBP-His) puncta having the same localization as the green⁺ (LC3B-GFP) puncta, based on the total red⁺ (HTTexon1-MBP-His) puncta.

9.2 Co-Localization Between mHTT and LC3B in the Striatal Cells of HD Mice Under the Influence of Compounds

The source of HD mouse striatal cells STHdh^{Q111/Q111} was as described above.

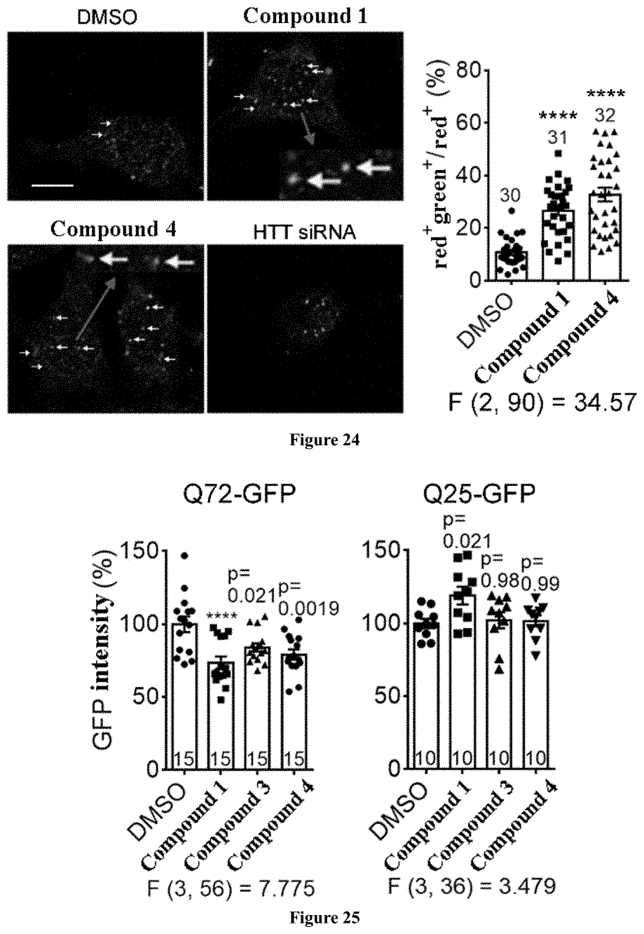

Using a method similar to 9.1, mHTT was detected by antibody 2166, and endogenous LC3B was detected by anti-LC3 antibody that specifically detects LC3B. Various fluorescent pixels were measured respectively and the co-localization between endogenous mHTT and LC3B in STHdhQ^{Q111/Q111} cells was analyzed (FIG. 24). The y-axis of the histogram in FIG. 24 showed the proportion of the red⁺ (HTTexon1-MBP-His) puncta having the same localization as the green⁺ (LC3B-GFP), based on the total red⁺ (HTTexon1-MBP-His) puncta. The compound obtained by the screening method of the present invention can promote the co-localization between the protein with abnormally expanded polyQ and LC3B in cells, which confirmed that the compound can simultaneously bind to the protein with abnormally expanded polyQ and LC3B to promote the enrichment of the protein with abnormally expanded polyQ to the cell substructures with autophagy function, thereby reducing the level of the protein with abnormally expanded polyQ in the cell through autophagy.

The inventor also unexpectedly discovered that common detectable markers such as GFP can be effectively used in the screening method of the present invention. Therefore, although the non-labeled detection methods, such as the unlabeled oblique-incidence reflectivity difference, have the advantage of not interfering with the detected object, the screening method of the present invention can also use other known detection methods, such as fluorescence.

### Example 10 Use of Proteins Containing Expanded Polyglutamine for Affinity Screening

10.1 Preparation of polyQ-GFP Protein

(1) PolyQ-GFP cDNA sequence (expressing Met-polyQ-GFP, wherein polyQ was Q72 or Q25) was synthesized de novo and subcloned into pcDNA vector. It was transfected into HEK293T cells (ATCC, CRL-3216) through forward transfection to obtain Q72-GFP expressing cells and Q25-GFP expressing cells, respectively. And the same method was used to obtain cells expressing Q53-GFP, cells expressing Q46-GFP, and cells expressing Q38-GFP.

(2) The polyQ-GFP proteins expressed by (1) were purified separately to obtain Q72-GFP, Q53-GFP, Q46-GFP, Q38-GFP and Q25-GFP (SEQ ID NO: 6). Among them, Q72-GFP, Q53-GFP, Q46-GFP, Q38-GFP differed from the amino acid sequence of SEQ ID NO: 6 in the length of the expanded polyglutamine.

### 10.2 Affinity Screening

To identify compound(s) that can selectively bind to proteins containing expanded polyglutamine, screening was conducted using a method similar to 1.1, except for that the target protein HTTexon1-Q72-MBP in step (1) of 1.1 was replaced with the polyQ-GFP protein prepared in 10.1, and the target protein HTTexon1-Q25-MBP in step (2) of 1.1 was replaced with GFP.

Among them, by analyzing the difference images of compound 1, compound 3, compound 4 after incubation and before incubation with polyQ-GFP protein (the image after incubation minus the image before incubation), it was found that these compounds did not bind to Q25-GFP or GFP, but binded to proteins with expanded polyglutamine. For example, compound 1, compound 3, and compound 4 showed certain binding affinity to proteins with 38 or more glutamine repeats. Using a method similar to 1.1, OI-RD was used to measure the affinity reaction parameters of compound 1, compound 3 and compound 4 with Q72-GFP, Q53-GFP, Q46-GFP and Q38-GFP. The results are shown in Tables 5-1 to 5-3.

TABLE 5-1
The association [symbol] rate constants, dissociation rate constants,
and equilibrium dissociation constants of the affinity reactions of
compound 1 with Q72-GFP, Q53-GFP, Q46-GFP, and Q38-GFP

| | Kₒₙ (min•nM)⁻¹ | K_{off} (min)⁻¹ | K_{d} (nM) |
|---|---|---|---|
| Q72-GFP | 8.37 × 10⁻⁵ | 1.51 × 10⁻² | 180.4 |
| Q53-GFP | 1.51 × 10⁻⁴ | 3.78 × 10⁻³ | 25.0 |
| Q46-GFP | 3.04 × 10⁻⁴ | 1.83 × 10⁻² | 60.2 |
| Q38-GFP | 1.06 × 10⁻⁵ | 2.03 × 10⁻² | 1915.1 |

TABLE 5-2
The association [symbol] rate constant [symbol] dissociation rate constants
and equilibrium dissociation constants of the affinity reactions of
compound 3 with Q72-GFP, Q53-GFP, Q46-GFP, and Q38-GFP

| | Kₒₙ (min•nM)⁻¹ | K_{off} (min)⁻¹ | K_{d} (nM) |
|---|---|---|---|
| Q72-GFP | 2.23 × 10⁻⁵ | 7.94 × 10⁻³ | 356.1 |
| Q53-GFP | 1.25 × 10⁻⁴ | 1.35 × 10⁻² | 108.0 |
| Q46-GFP | 1.29 × 10⁻⁴ | 7.67 × 10⁻³ | 59.5 |
| Q38-GFP | 8.72 × 10⁻⁶ | 2.62 × 10⁻² | 3004.6 |

TABLE 5-3
The association [symbol] rate constants, dissociation rate constants,
and equilibrium dissociation constants of the affinity reactions of
compound 4 with Q72-GFP, Q53-GFP, Q46-GFP, and Q38-GFP

| | Kₒₙ (min•nM)⁻¹ | K_{off} (min)⁻¹ | K_{d} (nM) |
|---|---|---|---|
| Q72-GFP | 6.20 × 10⁻⁵ | 1.92 × 10⁻² | 309.7 |
| Q53-GFP | 1.08 × 10⁻⁴ | 4.65 × 10⁻³ | 43.1 |
| Q46-GFP | 7.65 × 10⁻⁶ | 2.57 × 10⁻² | 3359.5 |
| Q38-GFP | 2.99 × 10⁻⁶ | 1.5 × 10⁻² | 5016.7 |

### 10.3 MST Detection of the Affinity of Compounds to Proteins Containing Expanded Polyglutamine

Following the method in 2.2, the affinity of compound 1, compound 3, compound 4 to the polyQ-GFP protein (Q25-GFP and Q72-GFP) was detected with a micro thermophoresis instrument for verification. The compound of the present invention showed no binding affinity to Q25-GFP, and the K_{d} of the compounds with Q72-GFP is shown in Table 6.

TABLE 6
The affinity reaction equilibrium dissociation
constant between the compound and the test protein

| K_{d} (μM) | compound 1 | compound 3 | compound 4 |
|---|---|---|---|
| Q72-GFP | 3.00 | 2.09 | 2.80 |

It can be seen from the above experiments that the compound of the present invention selectively binded to proteins containing expanded polyglutamine. A protein with abnormally expanded polyQ or a fragment thereof containing the polyQ part can be applied to the screening method of the present invention. The screening method of the present invention can be effectively applied to screening or identifying compound(s) for the treatment or prevention of diseases caused by proteins containing expanded polyglutamine, such as polyQ-related neurodegenerative disorders.

### Example 11 Effects of the Compounds on the Level of the Protein Containing Expanded Polyglutamine in the Cell

The Q72-GFP expressing cells and Q25-GFP expressing cells obtained in step (1) of 10.1 were used. The cells were treated with 100 nM compound 1, or 100 nM compound 3 or 50 nM compound 4 for 2 days. Then the level of polyQ-GFP was measured by Incucyte fluorescence counting (FIG. 25). The compounds obtained according to the screening method of the present invention can selectively reduce the level of proteins containing expanded polyglutamine in cells. It showed that the compounds obtained from screening had extended pharmacological effects and can be used to treat or prevent diseases caused by proteins containing expanded polyglutamine.

The following Examples 12 to 14 further test the effects of the compounds obtained by the screening method of the present invention on the levels of ATXN3 with abnormally expanded polyQ and ATXN1 with abnormally expanded polyQ, and it is further confirmed that the screening method of the present invention can use a protein containing an expanded polyglutamine as a target protein representing a disease state.

### Example 12 Using ATXN3 with Abnormally Expanded polyQ for Affinity Screening

### 12.1 Affinity Screening

Screening was conducted using a method similar to 1.1, except for that the target protein HTTexon1-Q72-MBP in step (1) of 1.1 was replaced with MBP-ATXN3-Q68 and the target protein HTTexon1-Q25-MBP in step (2) of 1.1 was replaced with MBP-ATXN3-Q28, to obtain compounds that can selectively bind to ATXN3 with abnormally expanded polyQ (MBP-ATXN3-Q68).

By analyzing the difference images of compound 1, compound 3, compound 4 after incubation and before incubation with MBP-ATXN3-Q68 and MBP-ATXN3-Q28 respectively (the image after incubation minus the image before incubation), it was found that these compounds did not bind to MBP-ATXN3-Q28, but binded to MBP-ATXN3-Q68.

12.2 MST Detection of the Affinity of the Compound to the ATXN with Abnormally Expanded polyQ

Following the method in 2.2, the affinity of the compound with MBP-ATXN3-Q28 and MBP-ATXN3-Q68 protein was verified by microscale thermophoresis.

The results showed that compound 1, compound 3 and compound 4 showed no binding affinity with normal ATXN3 protein (MBP-ATXN3-Q28), and the K_{d} of the compounds with abnormal ATXN3 protein (MBP-ATXN3-Q68) is shown in Table 7.

TABLE 7
The affinity reaction equilibrium dissociation
constant between the compound and the test protein

| K_{d} (μM) | compound 1 | compound 3 | compound 4 |
|---|---|---|---|
| MBP-ATXN3-Q68 | 6.32 | 1.02 | 2.77 |

### Example 13 Effects of Compounds on ATXN3 Level in Fibroblasts of Patients with Spinocerebellar Ataxia Type 3

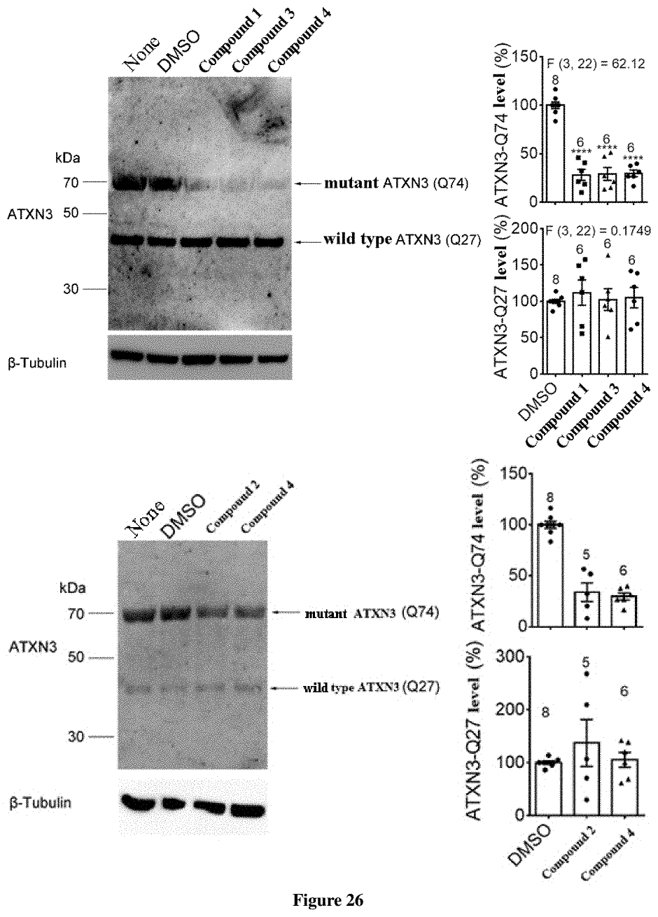

SCA3 patient fibroblasts (Q74), wild-type cells (Q27) were treated with 100 nM compound 1, or 100 nM compound 2, or 100 nM compound 3 or 50 nM compound 4 for 2 days. Then the levels of mutant ATXN3 protein (ATXN3-Q74, SEQ ID NO: 2) and wild-type ATXN3 protein (ATXN3-Q27, SEQ ID NO: 7) were determined by Western-blots (FIG. 26). A decrease in mutant ATXN3 protein level was observed on SCA3 patient fibroblasts (Q74), but no decrease in wild-type ATXN3 protein level was observed. Therefore, the compound of the present invention can be used to treat SCA3.

### Example 14 Using ATXN1 with Abnormally Expanded polyQ for Affinity Screening

14.1 Preparation of ATXN1 with Abnormally Expanded polyQ

(1) PolyQ-ATXN1 cDNA (where polyQ is Q92) was synthesized de novo and subcloned into pcDNA vector. It transfected into HEK293T cells (ATCC, CRL-3216) to obtain cells expressing His-ATXN1-Q92 (SEQ ID NO: 3 with a His tag at the N-terminal).

(2) The His-ATXN1-Q92 expressed in (1) was purified to obtain ATXN1 with abnormally expanded polyQ.

### 14.2 Affinity Screening

To identify compound(s) that can bind to ATXN1 with abnormally expanded polyQ, screening was conducted using a method similar to step (1) of 1.1, except for that the target protein HTTexon1-Q72-MBP in step (1) of 1.1 was replaced with His-ATXN1-Q92.

By analyzing the difference images of compound 1, compound 2, compound 3, compound 4 after incubation and before incubation with His-ATXN1-Q92 (the image after incubation minus the image before incubation), it was found that these compounds can bind to His-ATXN1-Q92.

### Example 15 Effects of Compounds on the Level of ATXN1 with Abnormally Expanded PolyQ in Cells

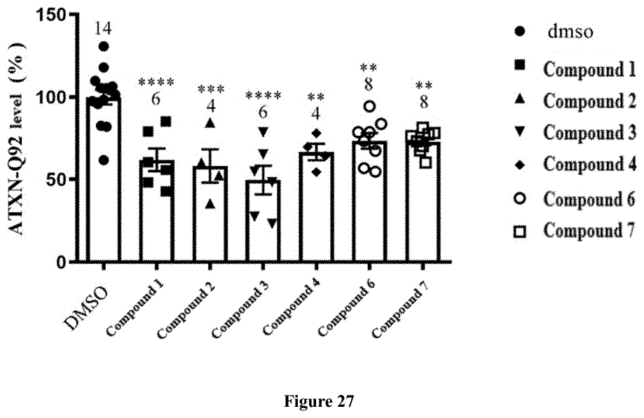

The His-ATXN1-Q92-expressing cells obtained in 14.1 were treated with 100 nM compound (compound 1, or compound 2, or compound 3, or compound 4, or compound 6, or compound 7) for 2 days. Then the level of mutant ATXN1 protein (His-ATXN1-Q92) was detected by HTRF (antibody pair: anti-His-Tb/MW1-D2, FIG. 27). Decreased levels of mutant ATXN1 were observed on fibroblasts (Q92) of SCA1 patients. Therefore, the compounds of the present invention can be used to treat or prevent spinocerebellar ataxia type 1.

In summary, it has been experimentally proved that the compounds obtained by the screening method of the present invention can reduce the levels of a variety of proteins containing expanded polyglutamine, such as the levels of mutant mHTT, mutant ATXN3, and mutant ATXN1. Therefore, the screening method of the present invention is effectively used for screening or identifying compound(s). Such compounds can be used to treat or prevent diseases caused by proteins containing expanded polyglutamine, such as polyQ-related neurodegenerative disorders.

Sequence Listing

[SEQ ID NO: 1] HTTexon1-Q72:
MATLEKLMKAFESLKSFQQQQQQQQQQQQQQQQQQQQQQQQQQQQQQQQQQQQ
QQQQQQQQQQQQQQQQQQQQQQQQQQQQQQQQQQQQPPPPPPPPPPPQLPQPPPQ
AQPLLPQPQPPPPPPPPPPGPAVAEEPLHR
[SEQ ID NO: 2] ATXN3-Q74:
MESIFHEKQEGSLCAQHCLNNLLQGEYFSPVELSSIAHQLDEEERMRMAEGGVTSED
YRTFLQQPSGNMDDSGFFSIQVISNALKVWGLELILFNSPEYQRLRIDPINERSFICNYK
EHWFTVRKLGKQWFNLNSLLTGPELISDTYLALFLAQLQQEGYSIFVVKGDLPDCEA
DQLLQMIRVQQMHRPKLIGEELAQLKEQRVHKTDLERMLEANDGSGMLDEDEEDLQ
RALALSRQEIDMEDEEADLRRAIQLSMQGSSRNISQDMTQTSGTNLTSEELRKRREAY
FEKQQQKQQQQQQQQQQQQQQQQQQQQQQQQQQQQQQQQQQQQQQQQQQQQQ
QQQQQQQQQQQQQQQQQQQQQQQQQQQQQRDLSGQSSHPCERPATSSGALGSDLG
KACSPFIMFATFTLYLT
[SEQ ID NO: 3] ATXN1-Q92:
MKSNQERSNECLPPKKRE1PATSRSSEEKAPTLPSDNHRVEGTAWLPGNPGGRGH
GGGRHGPAGTSVELGLQQGIGLHKALSTGLDYSPPSAPRSVPVATTLPAAYATPQPGTP
VSPVQYAHLPHTFQFIGSSQYSGTYASFIPSQLIPPTANPVTSAVASAAGATTPSQRSQL
EAYSTLLANMGSLSQTPGHKAEQQQQQQQQQQQQQQQQQQQQQQQQQQQQQQQQ
QQQQQQQQQQQQQQQQQQQQQQQQQQQQQQQQQQQQQQQQQQQQQQQQQQQQ
QQQQQQQQHQHQQQQQQQQQQQQQQHLSRAPGLITPGSPPPAQQNQYVHISSSPQN
TGRTASPPAIPVHLHPHQTMIPHTLTLGPPSQVVMQYADSGSHFVPREATKKAESSRLQ
QAIQAKEVLNGEMEKSRRYGAPSSADLGLGKAGGKSVPHPYESRHVVVHPSPSDYSS
RDPSGVRASVMVLPNSNTPAADLEVQQATHREASPSTLNDKSGLHLGKPGHRSYALS
PHTVIQTTHSASEPLPVGLPATAFYAGTQPPVIGYLSGQQQAITYAGSLPQHLVIPGTQP
LLIPVGSTDMEASGAAPAIVTSSPQFAAVPHTFVTTALPKSENFNPEALVTQAAYPAMV
QAQIHLPVVQSVASPAAAPPTLPPYFMKGSIIQLANGELKKVEDLKTEDFIQSAEISND
LKIDSSTVERIEDSHSPGVAVIQFAVGEHRAQVSVEVLVEYPFFVFGQGWSSCCPERTS
QLFDLPCSKLSVGDVCISLTLKNLKNGSVKKGQPVDPASVLLKHSKADGLAGSRHRY
AEQENGINQGSAQMLSENGELKFPE KMGLPAAPFLTKIEPSKPAATRKRRWSAPESRK
LEKSEDEPPLTLPKPSLIPQEVKICIEGRSNVGK
[SEQ ID NO: 4] HTT-Q23:
MATLEKLMKAFESLKSFQQQQQQQQQQQQQQQQQQQQQQQPPPPPPPPPPPQLPQPP
PQAQPLLPQPQPPPPPPPPPPGPAVAEEPLHRPKKELSATKKDRVNHCLTICENIVAQSV
RNSPEFQKLLGIAMELFLLCSDDAESDVRMVADECLNKVIKALMDSNLPRLQLELYK
EIKKNGAPRSLRAALWRFAELAHLVRPQKCRPYLVNLLPCLTRTSKRPEESVQETLAA
AVPKIMASFGNFANDNEIKVLLKAFIANLKSSSPTIRRTAAGSAVSICQHSRRTQYFYS
WLLNVLLGLLVPVEDEHSTLLILGVLLTLRYLVPLLQQQVKDTSLKGSFGVTRKEME
VSPSAEQLVQVYELTLHHTQHQDHNVVTGALELLQQLFRTPPPELLQTLTAVGGIGQL
TAAKEESGGRSRSGSIVELIAGGGSSCSPVLSRKQKGKVLLGEEEALEDDSESRSDVSS
SALTASVKDEISGELAASSGVSTPGSAGHDIITEQPRSQHTLQADSVDLASCDLTSSAT
DGDEEDILSHSSSQVSAVPSDPAMDLNDGTQASSPISDSSQTTTEGPDSAVTPSDSSEW
LDGTDNQYLGLQIGQPQDEDEEATGILPDEASEAFRNSSMALQQAHLLKNMSHCRQP
SDSSVDKFVLRDEATEPGDQENKPCRIKGDIGQSTDDDSAPLVHCVRLLSASFLLTGG
KNVLVPDRDVRVSVKALALSCVGAAVALHPESFFSKLYKVPLDTTEYPEEQYVSDILN
YIDHGDPQVRGATAILCGTLICSILSRSRFHVGDWMGTIRTLTGNTFSLADCIPLLRKTL
KDESSVTCKLACTAVRNCVMSLCSSSYSELGLQLIIDVLTLRNSSYWLVRTELLETLAE
IDFRLVSFLEAKAENLHRGAHHYTGLLKLQERVLNNVVIHLLGDEDPRVRHVAAASLI
RLVPKLFYKCDQGQADPVVAVARDQSSVYLKLLMHETQPPSHFSVSTITRIYRGYNLL
PSITDVTMENNLSRVIAAVSHELITSTTRALTFGCCEALCLLSTAFPVCIWSLGWHCGV
PPLSASDESRKSCTVGMATMILTLLSSAWFPLDLSAHQDALILAGNLLAASAPKSLRSS
WASEEEANPAATKQEEVWPALGDRALVPMVEQLFSHLLKVINICAHVLDDVAPGPAI
KAALPSLTNPPSLSPIRRKGKEKEPGEQASVPLSPKKGSEASAASRQSDTSGPVTTSKS
SSLGSFYHLPSYLKLHDVLKATHANYKVTLDLQNSTEKFGGFLRSALDVLSQILELAT
LQDIGKCVEEILGYLKSCFSREPMMATVCVQQLLKTLFGTNLASQFDGLSSNPSKSQG
RAQRLGSSSVRPGLYHYCFMAPYTHFTQALADASLRNMVQAEQENDTSGWFDVLQ
KVSTQLKTNLTSVTKNRADKNAIHNHIRLFEPLVIKALKQYTTTTCVQLQKQVLDLL
AQLVQLRVNYCLLDSDQVFIGFVLKQFEYIEVGQFRESEAIIPNIFFFLVLLSYERYHSK
QIIGIPKIIQLCDGIIVIASGRKAVTHAIPALQPIVHDLFVLRGTNKADAGKELETQKEVV
VSMLLRLIQYHQVLEMFILVLQQCHKENEDKWKRLSRQIADIILPMLAKQQMHIDSH
EALGVLNTLFEILAPSSLRPVDMLLRSMFVTPNTMASVSTVQLWISGILAILRVLISQST
EDIVLSRIQELSFSPYLISCTVINRLRDGDSTSTLEEHSEGKQIKNLPEETFSRFLLQLVG
ILLEDIVTKQLKVEMSEQQHTFYCQELGTLLMCLIHIFKSGMFRRITAAATRLFRSDGC
GGSFYTLDSLNLRARSMITTHPALVLLWCQILLLVNHTDYRWWAEVQQTPKRHSLSS
TKLLSPQMSGEEEDSDLAAKLGMCNREIVRRGALILFCDYVCQNLHDSEHLTWLIVN
HIQDLISLSHEPPVQDFISAVHRNSAASGLFIQAIQSRCENLSTPTMLKKTLQCLEGIHL
SQSGAVLTLYVDRLLCTPFRVLARMVDILACRRVEMLLAANLQSSMAQLPMEELNRI
QEYLQSSGLAQRHQRLYSLLDRFRLSTMQDSLSPSPPVSSHPLDGDGHVSLETVSPDK
DWYVHLVKSQCWTRSDSALLEGAELVNRIPAEDMNAFMMNSEFNLSLLAPCLSLGM
SEISGGQKSALFEAAREVTLARVSGTVQQLPAVHHVFQPELPAEPAAYWSKLNDLFGD
AALYQSLPTLARALAQYLVVVSKLPSHLHLPPEKEKDIVKFVVATLEALSWHLIHEQIP
LSLDLQAGLDCCCLALQLPGLWSVVSSTEFVTHACSLIYCVHFILEAVAVQPGEQLLSP
ERRTNTPKAISEEEEEVDPNTQNPKYITAACEMVAEMVESLQSVLALGHKRNSGVPAF
LTPLLRNIIISLARLPLVNSYTRVPPLVWKLGWSPKPGGDFGTAFPEIPVEFLQEKEVFK
EFIYRINTLGWTSRTQFEETWATLLGVLVTQPLVMEQEESPPEEDTERTQINVLAVQAI
TSLVLSAMTVPVAGNPAVSCLEQQPRNKPLKALDTRFGRKLSIIRGIVEQEIQAMVSKR
ENIATHHLYQAWDPVPSLSPATTGALISHEKLLLQINPERELGSMSYKLGQVSIHSVWL
GNSITPLREEEWDEEEEEEADAPAPSSPPTSPVNSRKHRAGVDIHSCSQFLLELYSRWI
LPSSSARRTPAILISEVVRSLLVVSDLFTERNQFELMYVTLTELRRVHPSEDEILAQYLV
PATCKAAAVLGMDKAVAEPVSRLLESTLRSSHLPSRVGALHGVLYVLECDLLDDTAK
QL1PVISDYLLSNLKGIAHCVNIHSQQHVLVMCATAFYLIENYPLDVGPEFSASIIQMC
GVMLSGSEESTPSIIYHCALRGLERLLLSEQLSRLDAESLVKLSVDRVNVHSPHRAMA
ALGLMLTCMYTGKEKVSPGRTSDPNPAAPDSESVIVAMERVSVLFDRIRKGFPCEARV
VARILPQFLDDFFPPQDIMNKVIGEFLSNQQPYPQFMATVVYKVFQTLHSTGQSSMVR
DWVMLSLSNFTQRAPVAMATWSLSCFFVSASTSPWVAAILPHVISRMGKLEQVDVNL
FCLVATDFYRHQIEEELDRRAFQSVLEVVAAPGSPYHRLLTCLRNVHKVTTC
[SEQ ID NO: 5] HTTexon1-Q25:
MATLEKLMKAFESLKSFQQQQQQQQQQQQQQQQQQQQQQQQQPPPPPPPPPPPQLP
QPPPQAQPLLPQPQPPPPPPPPPPGPAVAEEPLHR
[SEQ ID NO: 6] Q25-GFP:
MQQQQQQQQQQQQQQQQQQQQQQQQQMSKGEELFTGVVPILVELDGDVNGH
KFSVRGEGEGDATNGKLTLKFICTTGKLPVPWPTLVTTLTYGVQCFSRYPDHMKRHD
FFKSAMPEGYVQERTISFKDDGTYKTRAEVKFEGDTLVNRIELKGIDFKEDGNILGHK
LEYNFNSHNVYITADKQKNGIKANFKIRHNVEDGSVQLADHYQQNTPIGDGPVLLPD
NHYLSTQSVLSKDPNEKRDHMVLLEFVTAAGITHGGSG
[SEQ ID NO: 7] ATXN3-Q27:
MESIFHEKQEGSLCAQHCLNNLLQGEYFSPVELSSIAHQLDEEERMRMAEGGVTSED
YRTFLQQPSGNMDDSGFFSIQVISNALKVWGLELILFNSPEYQRLRIDPINERSFICNYK
EHWFTVRKLGKQWFNLNSLLTGPELISDTYLALFLAQLQQEGYSIFVVKGDLPDCEA
DQLLQMIRVQQMHRPKLIGEELAQLKEQRVHKTDLERMLEANDGSGMLDEDEEDLQ
RALALSRQEIDMEDEEADLRRAIQLSMQGSSRNISQDMTQTSGTNLTSEELRKRREAY
FEKQQQKQQQQQQQQQQQQQQQQQQQQQQQQQQQRDLSGQSSHPCERPATSSGAL
GSDLGKACSPFIMFATFTLYLT
[SEQ ID NO: 8] LC3B:
MPSEKTFKQRRTFEQRVEDVRLIREQHPTKIPVIIERYKGEKQLPVLDKTKFLVPDHVN
MSELIKIIRRRLQLNANQAFFLLVNGHSMVSVSTPISEVYESEKDEDGFLYMVYASQE
TFGMKLSV

1. A method for screening or identifying a compound for the treatment or prevention of a polyQ-related neurodegenerative disorder, comprising:
(I): contacting a candidate compound with a test system comprising:
(1) a protein with abnormally expanded polyQ or a fragment thereof containing the polyQ part, and
(2) a LC3B protein, a homologue thereof, or a fragment thereof; and
(II): determining the ability of the candidate compound to modulate the binding between (1) a protein with abnormally expanded polyQ or a fragment thereof containing the polyQ part, and (2) a LC3B protein, a homologue thereof, or a fragment thereof; and selecting a compound that positively modulates the binding.

2. The method of claim 1, wherein step (II) comprises:
respectively, in the presence/absence of the candidate compound, determining the binding affinity between (1) a protein with abnormally expanded polyQ or a fragment thereof containing the polyQ part, and (2) a LC3B protein, a homologue thereof, or a fragment thereof, and/or
respectively, in the presence/absence of the candidate compound, determining the binding amount of (1) a protein with abnormally expanded polyQ or a fragment thereof containing the polyQ part, and (2) a LC3B protein, a homologue thereof, or a fragment thereof.

3. The method of claim 2, wherein the protein with abnormally expanded polyQ comprises a polyQ part with polyQ length≥19.

4. The method of claim 3, wherein (2) a LC3B protein, a homologue thereof, or a fragment thereof comprises a sequence having 90% or higher sequence identity with the amino acid sequence of SEQ ID NO: 8.

5. The method of claim 4, wherein the method for determining the binding is selected from the group consisting of in vitro pull-down test, Split-TEV, co-immunoprecipitation, affinity chromatography, complex co-purification, enzyme-linked immunosorbent assay, fluorescent molecular sieve, yeast two-hybrid, HIP-HOP, time-resolved fluorescence resonance energy transfer, chemiluminescence method, surface plasmon resonance, isothermal titration calorimetry, micro thermophoresis and any combination thereof.

6. The method of claim 5, further comprising a step of pre-screening the candidate compound for affinity before step (I), comprising:
(A): detecting the binding affinity of the candidate compound to (1) a protein with abnormally expanded polyQ or a fragment thereof containing the polyQ part, and/or
(B): detecting the binding affinity of the candidate compound to (2) a LC3B protein, a homologue thereof, or a fragment thereof; and
(C): selecting the compound having binding affinity to (1) a protein with abnormally expanded polyQ or a fragment thereof containing the polyQ part and/or (2) a LC3B protein, a homologue thereof, or a fragment thereof;
wherein step (A) and step (B) can be carried out independently in any order.

7. The method of claim 6, wherein:
step (A) comprises:
determining the affinity reaction equilibrium dissociation constant of the candidate compound and (1) a protein with abnormally expanded polyQ or a fragment thereof containing the polyQ part; and
selecting a compound whose affinity reaction equilibrium dissociation constant is 100 μM or less; and/or
step (B) comprises:
determining the affinity reaction equilibrium dissociation constant of the candidate compound compound(s) and (2) a LC3B protein, a homologue thereof, or a fragment thereof; and
selecting the compound whose affinity reaction equilibrium dissociation constant is 100 μM or less.

8. The method of claim 7, wherein the method for determining binding affinity is selected from the group consisting of proximity scintillation analysis, fluorescence resonance energy transfer, fluorescence polarization detection, fluorescent molecular sieves, micro thermophoresis, chemiluminescence, surface plasmon resonance, isothermal titration calorimetry, oblique-incidence reflectivity difference and any combination thereof.

9. The method of claim 8, wherein the step of pre-screening the candidate compound for affinity is performed by high-throughput screening using the oblique-incidence reflectivity difference; comprising:
(a) immobilizing the candidate compound to prepare a compound chip, and scanning the chip to obtain the image before incubation;
(b) incubating the compound chip with the target protein, and scanning the chip to obtain the image after incubation; wherein the target protein is (1) a protein with abnormally expanded polyQ or a fragment thereof containing the polyQ part or (2) a LC3B protein, a homologue thereof, or a fragment thereof; and
(c) analyzing the difference image (the image after incubation minus the image before incubation); and selecting the compound that can bind to both (1) a protein with abnormally expanded polyQ or a fragment thereof containing the polyQ part, and (2) a LC3B protein, a homologue thereof, or a fragment thereof.

10. The method of claim 9, further comprising the following steps:
(D): detecting the binding of the candidate compound to a normal polyQ protein or a fragment thereof containing the polyQ part; and selecting the compound that does not bind to the normal polyQ protein or the fragment thereof containing the polyQ part.

11. A method for the treatment or prevention of a polyQ-related neurodegenerative disorder, the method comprising administering an effective amount of a compound identified by the method of claim 1, or a pharmaceutical composition thereof, to a subject with the polyQ-related neurodegenerative disorder.

12. The method of claim 11, wherein the polyQ-related neurodegenerative disorder is selected from the group consisting of spinocerebellar ataxia type 1, spinocerebellar ataxia type 2, spinocerebellar ataxia type 3, spinocerebellar ataxia type 7, spinocerebellar ataxia type 12 and spinocerebellar ataxia type 17.

13. The method of claim 11, wherein the polyQ-related neurodegenerative disorder is selected from the group consisting of dentatorubral-pallidoluysian atrophy, Huntington's disease and spinal-bulbar muscular atrophy.

14. The method of claim 11, wherein the detection includes the step of analyzing a target sample obtained from said subject, comprising:
i) labeling the compound with a detectable label such as a fluorescent group;
ii) treating the target sample with the compound obtained in i); and
iii) detecting the position and/or intensity of the detectable signal in the target sample; and obtaining the position and/or content of the protein with abnormally expanded polyQ or the fragment thereof containing the polyQ part in the target sample.

15. A method for the treatment or prevention of a polyQ-related neurodegenerative disorder, the method comprising administering an effective amount of a compound or a pharmaceutical composition thereof to a subject with the polyQ-related neurodegenerative disorder, wherein the compound is selected from the group consisting of: 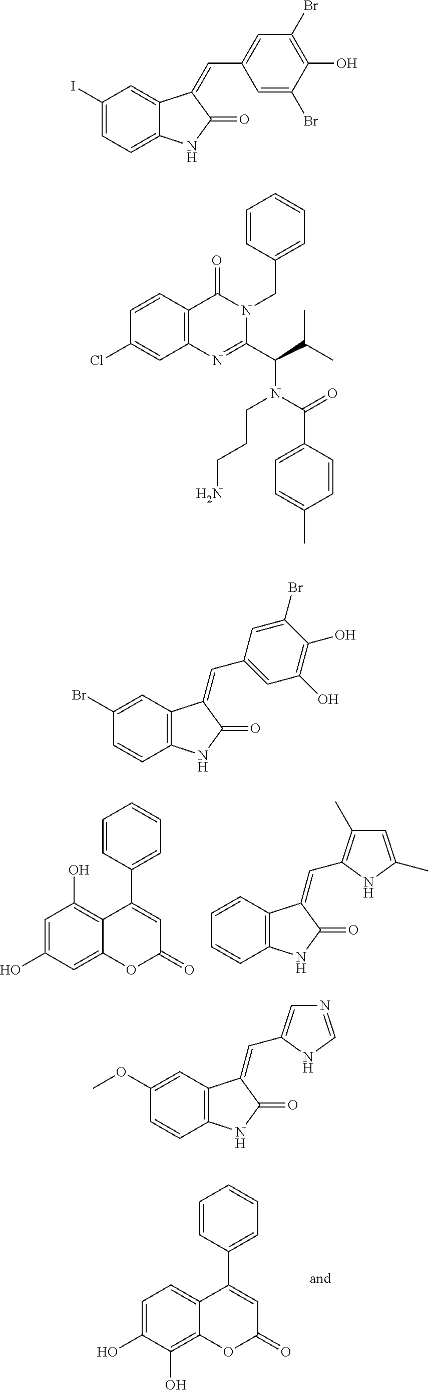 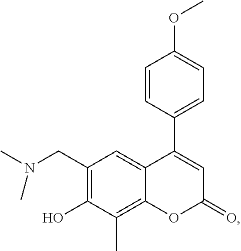 [SMILES: COc1ccc(-c2cc(=O)oc3c(C)c(O)c(CN(C)C)cc23)cc1]
or pharmaceutically acceptable salt thereof.

16. A pharmaceutical composition, detection reagent or kit comprising a compound obtained by the method of claim 1.

17. A pharmaceutical composition, detection reagent, kit or screening system, which comprises at least one compound selected from the group consisting of: 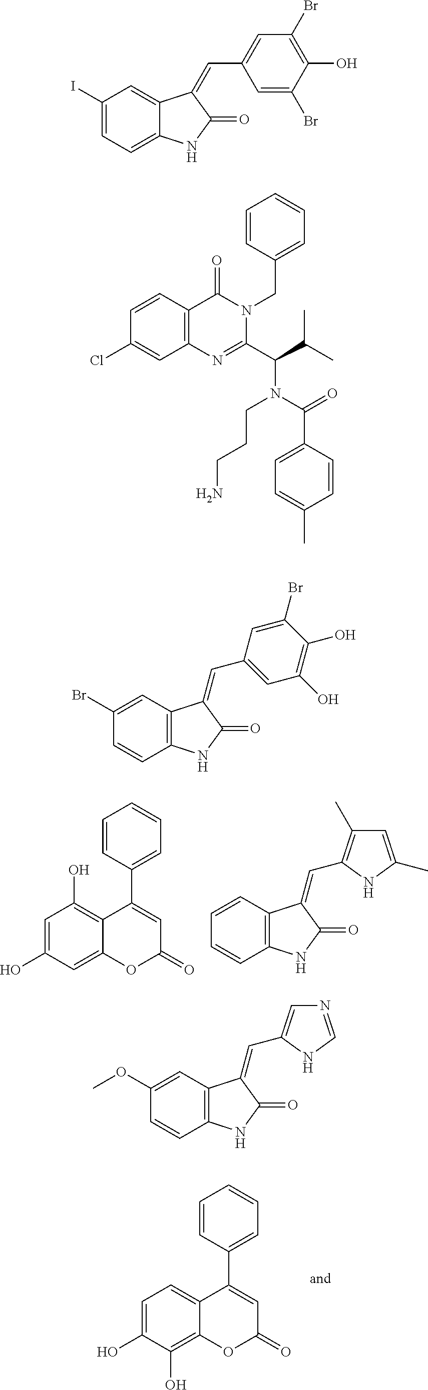 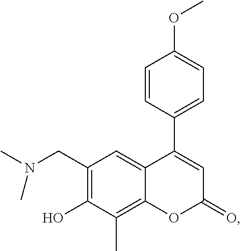 [SMILES: COc1ccc(-c2cc(=O)oc3c(C)c(O)c(CN(C)C)cc23)cc1]
or a pharmaceutical composition thereof.

18. A screening system, comprising:
(1) a protein with abnormally expanded polyQ or a fragment thereof containing the polyQ part, and
(2) a LC3B protein, a homologue thereof, or a fragment thereof.

19. A method for the treatment or prevention of a polyQ-related neurodegenerative disorder, the method comprising administering a compound or a pharmaceutical composition to a subject with the polyQ-related neurodegenerative disorder, wherein the compound or pharmaceutical composition positively modulates the binding between (1) a protein with abnormally expanded polyQ or a fragment thereof containing the polyQ part, and (2) a LC3B protein, a homologue thereof, or a fragment thereof.


SEQUENCE LISTING (8 sequences)

SEQ ID NO: 1 - protein, 138 aa, Homo sapiens
  HTTexon1-Q72
MATLEKLMKAFESLKSFQQQQQQQQQQQQQQQQQQQQQQQQQQQQQQQQQQQQQQQQQQQQQQQQQQQQQQQQQQQQQQQQQQQQQQQQPPPPPPPPPPPQLPQPPPQAQPLLPQPQPPPPPPPPPPGPAVAEEPLHR

SEQ ID NO: 2 - protein, 412 aa, Homo sapiens
  ATXN3-Q74
MESIFHEKQEGSLCAQHCLNNLLQGEYFSPVELSSIAHQLDEEERMRMAEGGVTSEDYRTFLQQPSGNMDDSGFFSIQVISNALKVWGLELILFNSPEYQRLRIDPINERSFICNYKEHWFTVRKLGKQWFNLNSLLTGPELISDTYLALFLAQLQQEGYSIFVVKGDLPDCEADQLLQMIRVQQMHRPKLIGEELAQLKEQRVHKTDLERMLEANDGSGMLDEDEEDLQRALALSRQEIDMEDEEADLRRAIQLSMQGSSRNISQDMTQTSGTNLTSEELRKRREAYFEKQQQKQQQQQQQQQQQQQQQQQQQQQQQQQQQQQQQQQQQQQQQQQQQQQQQQQQQQQQQQQQQQQQQQQQQQQQQQQQRDLSGQSSHPCERPATSSGALGSDLGKACSPFIMFATFTLYLT

SEQ ID NO: 3 - protein, 895 aa, Homo sapiens
  ATXN1-Q92
MKSNQERSNECLPPKKREIPATSRSSEEKAPTLPSDNHRVEGTAWLPGNPGGRGHGGGRHGPAGTSVELGLQQGIGLHKALSTGLDYSPPSAPRSVPVATTLPAAYATPQPGTPVSPVQYAHLPHTFQFIGSSQYSGTYASFIPSQLIPPTANPVTSAVASAAGATTPSQRSQLEAYSTLLANMGSLSQTPGHKAEQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQHQHQQQQQQQQQQQQQQHLSRAPGLITPGSPPPAQQNQYVHISSSPQNTGRTASPPAIPVHLHPHQTMIPHTLTLGPPSQVVMQYADSGSHFVPREATKKAESSRLQQAIQAKEVLNGEMEKSRRYGAPSSADLGLGKAGGKSVPHPYESRHVVVHPSPSDYSSRDPSGVRASVMVLPNSNTPAADLEVQQATHREASPSTLNDKSGLHLGKPGHRSYALSPHTVIQTTHSASEPLPVGLPATAFYAGTQPPVIGYLSGQQQAITYAGSLPQHLVIPGTQPLLIPVGSTDMEASGAAPAIVTSSPQFAAVPHTFVTTALPKSENFNPEALVTQAAYPAMVQAQIHLPVVQSVASPAAAPPTLPPYFMKGSIIQLANGELKKVEDLKTEDFIQSAEISNDLKIDSSTVERIEDSHSPGVAVIQFAVGEHRAQVSVEVLVEYPFFVFGQGWSSCCPERTSQLFDLPCSKLSVGDVCISLTLKNLKNGSVKKGQPVDPASVLLKHSKADGLAGSRHRYAEQENGINQGSAQMLSENGELKFPEKMGLPAAPFLTKIEPSKPAATRKRRWSAPESRKLEKSEDEPPLTLPKPSLIPQEVKICIEGRSNVGK

SEQ ID NO: 4 - protein, 3144 aa, Homo sapiens
  HTT-Q23
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 5 - protein, 91 aa, Homo sapiens
  HTTexon1-Q25
MATLEKLMKAFESLKSFQQQQQQQQQQQQQQQQQQQQQQQQQPPPPPPPPPPPQLPQPPPQAQPLLPQPQPPPPPPPPPPGPAVAEEPLHR

SEQ ID NO: 6 - protein, 261 aa, Homo sapiens
  Q25-GFP
MQQQQQQQQQQQQQQQQQQQQQQQQQMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGGSG

SEQ ID NO: 7 - protein, 365 aa, Homo sapiens
  ATXN3-Q27
MESIFHEKQEGSLCAQHCLNNLLQGEYFSPVELSSIAHQLDEEERMRMAEGGVTSEDYRTFLQQPSGNMDDSGFFSIQVISNALKVWGLELILFNSPEYQRLRIDPINERSFICNYKEHWFTVRKLGKQWFNLNSLLTGPELISDTYLALFLAQLQQEGYSIFVVKGDLPDCEADQLLQMIRVQQMHRPKLIGEELAQLKEQRVHKTDLERMLEANDGSGMLDEDEEDLQRALALSRQEIDMEDEEADLRRAIQLSMQGSSRNISQDMTQTSGTNLTSEELRKRREAYFEKQQQKQQQQQQQQQQQQQQQQQQQQQQQQQQQRDLSGQSSHPCERPATSSGALGSDLGKACSPFIMFATFTLYLT

SEQ ID NO: 8 - protein, 125 aa, Homo sapiens
  LC3B
MPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMKLSV